(19) United States
(12) Patent Application Publication
(10) Pub. No.: US 2026/0183533 A1
(43) Pub. Date: Jul. 2, 2026

(54) CIRCULATORY ASSIST DEVICE WITH VASCULAR LUMEN SEALING

(71) Applicant: CardiacBooster B.V., Nijmegen (NL)

(72) Inventors: Florian Niklas Ludwig, Hilversum (NL); Daniël Immanuel Michaël van Dort, Nijmegen (NL); Patrick Griffin, Galway (IE)

(21) Appl. No.: 19/546,039

(22) Filed: Feb. 20, 2026

Related U.S. Application Data
(63) Continuation of application No. PCT/IB2024/058253, filed on Aug. 24, 2024.
(60) Provisional application No. 63/578,512, filed on Aug. 24, 2023.

(51) Int. Cl. A61M 60/90; A61M 39/24; A61M 60/13; A61M 60/216; A61M 60/295; A61M 60/81; A61M 60/892; A61M 60/896

(52) U.S. Cl. CPC A61M 60/90; A61M 39/24; A61M 60/13; A61M 60/216; A61M 60/295; A61M 60/81; A61M 60/892; A61M 60/896; A61M 2039/242; A61M 2039/2433; A61M 2205/0266; A61M 2205/3331; A61M 2210/125; A61M 2210/127

(57) ABSTRACT

Circulatory assist devices and methods are disclosed herein. In some variations, a circulatory assist system may be configured for placement in a cardiovascular lumen (e.g., aorta) and include a pump arrangement comprising a pump region configured to receive blood, an outflow region, and a pump in the pump region and configured to drive the received blood toward the outflow region. The circulatory assist system may further include a vascular seal configured to form a peripheral seal between the pump arrangement and an inner wall of the cardiovascular lumen.

### CROSS-REFERENCE TO RELATED APPLICATIONS

The present application is a continuation of International Application No. PCT/IB2024/058253, filed Aug. 24, 2024, which claims the benefit of priority to U.S. Provisional Patent Application No. 63/578,512 , filed Aug. 24, 2023, which is incorporated by reference herein in its entirety.

### TECHNICAL FIELD

The present technology relates to a circulatory assist device.

### BACKGROUND

For patients suffering from cardiogenic shock, or those undergoing high-risk percutaneous coronary interventions (PCI), a patient's heart function may be compromised such that the use of circulatory assist devices may be required to maintain adequate blood flows through the circulatory system. Although there is some variation depending on patient size and condition, circulatory assist devices for patients undergoing high risk PCI typically must produce blood flows of least 3 L/min to maintain adequate circulation, while for patients in cardiogenic shock, a minimum of 5 L/min is generally considered necessary.

One type of circulatory assist device is transvalvular percutaneous mechanical cardiac support devices (pMCS), which are configured to be placed across the aortic valve. The placement of such transvalvular devices, however, carries the risk of endovascular or valvular injury, and so typically must be performed by a highly trained and skilled interventional cardiologist who can successfully navigate the devices over the aortic arch and across the aortic valve without incurring tissue damage. However, this level of skill is not always present in interventional cardiologists who may be inexperienced. Additionally, the clinical need for circulatory assist devices may arise in situations in which skilled operators are not available (e.g., emergency situations, such as in an ambulance or peripheral acute chest pain intervention units).

What is needed, therefore, are improved circulatory support systems and methods.

### SUMMARY

The present technology is illustrated, for example, according to various aspects described below, including with reference to FIGS. 1A-19E. Various examples of aspects of the present technology are described as numbered Examples (1, 2, 3, etc.) for convenience. These are provided as Examples and do not limit the present technology.

Example A1. A blood pump system configured for placement in a vascular lumen, the blood pump system comprising:
a pump arrangement comprising an outflow region and configured to receive blood and drive the received blood toward the outflow region; and
a vascular seal configured to form a peripheral seal between the pump arrangement and an inner wall of the vascular lumen upstream of the outflow region.

Example A2. The blood pump system of Example A1, wherein the vascular seal is operable in a closed state in which the peripheral seal is formed with the inner wall, and an open state in which blood may flow between the pump arrangement and the inner wall.

Example A3. The blood pump system of Example A2, wherein:
the vascular seal is in the closed state when pressure on a downstream side of the vascular seal is greater than pressure on an upstream side of the vascular seal, and
the vascular seal is in the open state when pressure on the upstream side of the vascular seal is greater than pressure on the downstream side of the vascular seal.

Example A4. The blood pump system of Example A2 or A3, further comprising an actuator configured to actively transition the vascular seal between the closed state and the open state.

Example A5. The blood pump system of any one of Examples A2-A4, wherein the vascular seal is radially expanded in the closed state and the vascular seal is radially contracted in the open state.

Example A6. The blood pump system of any one of Examples A1-A5, wherein the pump arrangement comprises a pump body with a conduit.

Example A7. The blood pump system of Example A6, wherein the vascular seal is arranged around a periphery of the pump body.

Example A8. The blood pump system of Example A7, wherein the vascular seal comprises a plurality of segments configured to conform to the inner wall of the vascular lumen.

Example A9. The blood pump system of any of Examples A1-A8, wherein the vascular seal is located between an inflow region of the pump arrangement and the outflow region.

Example A10. The blood pump system of Example A6 or A9, wherein the conduit comprises a membrane.

Example A11. The blood pump system of Example A10, wherein the conduit further comprises an expandable support, wherein the membrane is adjacent to a surface of the expandable support.

Example A12. The blood pump system of any one of Examples A1-A11, wherein the pump arrangement further comprises an inlet valve configured to convey blood to the pump region.

Example A13. The blood pump system of any one of Examples A1-A12, wherein the pump arrangement further comprises an outlet valve configured to convey blood from the outflow region.

Example A14. The blood pump system of any one of Examples A1-A13, wherein the pump arrangement comprises an impeller pump.

Example A15. The blood pump system of any one of Examples A1-A13, wherein the pump arrangement comprises a volume displacement member.

Example A16. The blood pump system of Example A15, wherein the volume displacement member comprises a balloon.

Example A17. The blood pump system of Example A16, further comprising:
an inflation member in fluidic communication with the balloon; and
a pump configured to cyclically operate the balloon in an expansion phase and a contraction phase.

Example A18. The blood pump system of any one of Examples A1-A17, wherein the outflow region comprises a compliant chamber with one or more outlets.

Example A19. The blood pump system of any one of Examples A1-A18, wherein the vascular seal is configured to extend radially outward from the pump region of the pump arrangement.

Example A20. The blood pump system of any one of Examples A1-A19, wherein the vascular seal is configured to extend radially outward from the outflow region of the pump arrangement.

Example A21. The blood pump system of any one of Examples A1-A20, wherein the vascular seal comprises a flexible membrane.

Example A22. The blood pump system of Example A21, wherein the vascular seal comprises one or more reinforcement members coupled to the membrane.

Example A23. The blood pump system of Example A22, wherein the one or more reinforcement members comprises at least one inflatable reinforcement member.

Example A24. The blood pump system of Example A22 or A23, wherein the one or more reinforcement members comprise a shape memory material.

Example A25. The blood pump system of any one of Examples A21-A24, wherein the membrane comprises one or more flap valves.

Example A26. The blood pump system of any one of Examples A1-A25, wherein the vascular seal comprises a skirt structure.

Example A27. The blood pump system of any one of Examples A1-26, wherein the vascular seal comprises a valve.

Example A28. The blood pump system of any one of Examples A1-A27, wherein the vascular seal comprises one or more leaflets.

Example A29. The blood pump system of any one of Examples A1-A28, wherein the outflow region is radially expandable to a bulbous shape to form the vascular seal.

Example A30. The blood pump system of any one of Examples A1-A29, further comprising one or more tethers configured to control the vascular seal.

Example A31. The blood pump system of any one of Examples A1-A30, wherein the vascular seal is a first vascular seal, and the blood pump system further comprises a second vascular seal arranged to form a second peripheral seal between the pump arrangement and the inner wall of the vascular lumen.

Example A32. The blood pump system of Example A31, further comprising a conduit containing at least one of the first and second vascular seals.

Example A33. The blood pump system of any one of Examples A1-A32, wherein the vascular lumen is in a descending aorta.

Example A34. The blood pump system of any one of Examples A1-A32, wherein the vascular lumen is in a pulmonary artery.

Example B1. A method comprising:
positioning a pump device in a vascular lumen of a patient, the pump device comprising:
   a pump arrangement with an outflow region; and
   a vascular seal arranged adjacent the pump arrangement;
forming a peripheral seal between the pump arrangement and an inner wall of the vascular lumen with the vascular seal; and
operating the pump arrangement to receive blood and drive the received blood through the outflow region and into the vascular lumen downstream of the peripheral seal.

Example B2. The method of Example B1, wherein positioning the pump device in a vascular lumen comprises positioning the pump device in a descending aorta of the patient.

Example B3. The method of Example B2, further comprising, after operating the pump device in a descending aorta of the patient at a first level of circulatory support, repositioning the pump device at least partially in a left ventricle of the patient and operating the pump device at a second level of circulatory support.

Example B4. The method of Example B3, further comprising, after operating the pump device in the left ventricle of the patient at the second level of the circulatory support, repositioning the pump device in the descending aorta of the patient and operating the pump device at a third level of circulatory support.

Example B5. The method of Example B1, wherein positioning the pump device in a vascular lumen comprises positioning the pump device at least partially in a left ventricle of the patient.

Example B6. The method of Example B5, further comprising, after operating the pump device in the left ventricle at a first level of circulatory support, repositioning the pump device in a descending aorta of the patient and operating the pump device at a second level of circulatory support.

Example B7. The method of Example B1, wherein positioning the pump device in a vascular lumen comprises positioning the pump device in a pulmonary artery of the patient.

Example B8. The method of Example B7, further comprising, after operating the pump device in the pulmonary artery at a first level of circulatory support, repositioning the pump device at least partially in a right ventricle of the patient and operating the pump device at second level of circulatory support.

Example B9. The method of Example B8, further comprising, after operating the pump device in the right ventricle, repositioning the pump device in the pulmonary artery of the patient and operating the pump device at a third level of circulatory support.

Example B10. The method of Example B1, wherein positioning the pump device in a vascular lumen comprises positioning the pump device at least partially in a right ventricle of the patient.

Example B11. The method of Example B10, further comprising, after operating the pump device in the right ventricle at a first level of circulatory support, repositioning the pump device at least partially in a pulmonary artery of the patient at a second level of circulatory support.

Example B12. The method of any one of Examples B1-B11, wherein the vascular seal is operable in a closed state and an open state.

Example B13. The method of Example B12, wherein the method comprises:
allowing the vascular seal to operate in the closed state when pressure on a downstream side of the vascular seal is greater than pressure in an upstream side of the vascular seal, and
allowing the vascular seal to operate in the open state when pressure on the upstream side of the vascular seal is greater than pressure in the downstream side of the vascular seal.

Example B14. The method of Example B12 or B13, wherein the method comprises actuating the vascular seal to transition the vascular seal between the closed state and the open state

Example B15. The method of any one of Examples B1-B14, wherein forming a peripheral seal comprises allowing the vascular seal to expand to form the peripheral seal between the pump arrangement and the inner wall of the vascular lumen.

Example B16. The method of Example B15, wherein allowing the vascular seal to expand comprises allowing the vascular seal to self-expand.

Example B17. The method of Example B15, wherein allowing the vascular seal to expand comprises actuating the vascular seal to expand.

Example B18. The method of any one of Examples B1-B17, wherein the vascular seal comprises a valve.

Example B19. The method of any one of Examples B1-B18, wherein the vascular seal comprises a skirt structure.

Example B20. The method of any one of Examples B1-B19, wherein the pump region comprises a pump body with a conduit.

Example B21. The method of any one of Examples B1-B20, wherein the pump region is defined at least partially by the vascular seal.

Example B22. The method of Example B21, wherein the vascular seal is a first vascular seal, and wherein the pump arrangement is further defined at least partially by a second vascular seal configured to form a second peripheral seal between the pump arrangement and the inner wall of the vascular lumen.

Example B23. The method of any one of Examples B1-B22, wherein the pump arrangement comprises an impeller pump.

Example B24. The method of any one of Examples B1-B22, wherein the pump arrangement comprises a volume displacement member.

Example B25. The method of Example B24, wherein the volume displacement member comprises a balloon.

Example B26. The method of any one of Examples B1-B25, further comprising radially expanding the flow of blood as the blood exits the outflow region.

Example B27. The method of any one of Examples B1-B26, further comprising actuating one or more actuation members to radially contract the vascular seal, and withdrawing the pump device from the vascular lumen of the patient.

Example B28. The method of any one of Examples B1-B27, wherein positioning the pump device in the vascular lumen of the patient is performed without imaging guidance.

Example C1. A blood pump system configured for placement in a vascular lumen, the blood pump system comprising:
a pump arrangement comprising:
a conduit with a pump region configured to receive blood and an outflow region; and
a pump in the conduit and configured to drive the received blood through the outflow region,
wherein at least a portion of the pump arrangement is configured to form a peripheral seal with an inner wall of the vascular lumen.

Example C2. The blood pump system of Example C1, wherein the outflow region has an outflow diameter larger than a diameter of the pump region.

Example C3. The blood pump system of Example C1 or C2, wherein the outflow region has a bulbous or flared shape.

Example C4. The blood pump system of Example C2 or C3, wherein the outflow region is configured to form the peripheral seal with the inner wall of the vascular lumen.

Example C5. The blood pump system of any one of Examples C2-C4, wherein the outflow region is expandable from a contracted shape to an expanded shape having the outflow diameter.

Example C6. The blood pump system of Example C5, wherein the outflow region is self-expandable from the contracted shape to the expanded shape.

Example C7. The blood pump system of Example C5, wherein the outflow region is configured to expand when blood is flowing through the outflow region.

Example C8. The blood pump system of any one of Examples C1-C7, wherein the conduit comprises at least one inlet region.

Example C9. The blood pump system of Example C8, further comprising at least one one-way valve disposed in the inlet region.

Example C10. The blood pump system of any one of Examples C1-C9, wherein the conduit comprises one or more radially-directed outlets.

Example C11. The blood pump system of Example C9, wherein the conduit comprises a circumferential array of radially-directed outlets.

Example C12. The blood pump system of any one of Examples C1-C11, wherein the conduit comprises a membrane.

Example C13. The blood pump system of Example C12, wherein the conduit further comprises an expandable support, wherein the membrane is adjacent to a surface of the expandable support.

Example C14. The blood pump system of any one of Examples C1-C13, further comprising a vascular seal configured to form the peripheral seal between the pump arrangement and an inner wall of the vascular lumen.

Example C15. The blood pump system of Example C14, wherein the vascular seal comprises a plurality of segments configured to conform to the inner wall of the vascular lumen.

Example C16. The blood pump system of any of Examples C1-C15, wherein the vascular seal is located between an inflow region of the pump arrangement and the outflow region.

Example C17. The blood pump system of Example C16, wherein the vascular seal is operable in a closed state and an open state.

Example C18. The blood pump system of Example C17, wherein:
the vascular seal is in the closed state when pressure on a downstream side of the vascular seal is greater than pressure on an upstream side of the vascular seal, and
the vascular seal is in the open state when pressure on the upstream side of the vascular seal is greater than pressure on the downstream side of the vascular seal.

Example C19. The blood pump system of Example C17 or C18, further comprising an actuator configured to actively transition the vascular seal between the closed state and the open state.

Example C20. The blood pump system of any one of Examples C14-C19, wherein the vascular seal comprises a valve.

Example C21. The blood pump system of any one of Examples C14-C20, wherein the vascular seal comprises a skirt structure.

Example C22. The blood pump system of any one of Examples C14-C21, wherein the vascular seal comprises a flexible membrane.

Example C23. The blood pump system of Example C22, wherein the vascular seal comprises one or more reinforcement members coupled to the membrane.

Example C24. The blood pump system of Example C23, wherein the one or more reinforcement members comprises at least one inflatable reinforcement member.

Example C25. The blood pump system of Example C23 or C24, wherein the one or more reinforcement members comprise a shape memory material.

Example C26. The blood pump system of any one of Examples C1-C25, wherein the pump comprises an impeller pump.

Example C27. The blood pump system of any one of Examples C1-C25, wherein the pump comprises a volume displacement member.

Example C28. The blood pump system of Example C27, wherein the volume displacement member comprises a balloon.

Example C29. The blood pump system of any one of Examples C1-C28, wherein the vascular lumen is in a descending aorta.

Example C30. The blood pump system of any one of Examples C1-C28, wherein the vascular lumen is in a pulmonary artery.

Example D1. A method comprising:
positioning a pump device in a vascular lumen of a patient, the pump device comprising a pump arrangement with a conduit having a pump region configured to receive blood and an outflow region, and a pump in the conduit;
operating the pump to drive received blood toward the outflow region; and
engaging an inner wall of the vascular lumen with a sealing portion of the pump device to form a peripheral seal around the pump device.

Example D2. The method of Example D1, wherein the pump device further comprises a vascular seal, wherein the method further comprises forming the peripheral seal between the pump arrangement and an inner wall of the vascular lumen with the vascular seal.

Example D3. The method of Example D1 or D2, wherein the vascular seal comprises the sealing portion of the pump device having an expanded diameter larger than a remaining portion of the pump device.

Example D4. The method of any one of Examples D1-D3 wherein the sealing portion is adjacent the outflow region of the pump device.

Example D5. The method of Example D3 or D4, further comprising expanding the vascular seal from a contracted diameter to the expanded diameter.

Example D6. The method of Example D5, wherein the vascular seal self-expands from the contracted diameter to the expanded diameter.

Example D7. The method of any one of Examples D3-D6, wherein the vascular seal expands to the expanded diameter when blood is flowing through the outflow region.

Example D8. The method of any one of Examples D2-D7, wherein forming a peripheral seal comprises allowing the vascular seal to expand to form the peripheral seal between the pump arrangement and the inner wall of the vascular lumen.

Example D9. The method of Example D8, wherein allowing the vascular seal to expand comprises allowing the vascular seal to self-expand.

Example D10. The method of Example D8, wherein allowing the vascular seal to expand comprises actuating the vascular seal to expand.

Example D11. The method of any one of Examples D1-D10, wherein the pump comprises an impeller pump.

Example D12. The method of any one of Examples D1-D10, wherein the pump comprises a volume displacement member.

Example D13. The method of Example D12, wherein the volume displacement member comprises a balloon.

Example D14. The method of any one of Examples D1-D13, further comprising actuating one or more tethers to radially contract the vascular seal, and withdrawing the pump device from the vascular lumen of the patient.

Example D15. The method of any one of Examples D1-D14, wherein positioning the pump device in the vascular lumen of the patient is performed without imaging guidance.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is an illustrative schematic of an example circulatory assist device in a cardiovascular lumen, in accordance with the present technology.

FIG. 1B is an illustrative schematic of an example circulatory assist device, in accordance with the present technology.

FIG. 1C is an illustrative schematic of an example circulatory assist device, in accordance with the present technology.

FIGS. 2A and 2B are illustrative schematics of an example circulatory assist device in active and passive states, in accordance with the present technology.

FIG. 3 is an illustrative schematic of a detailed view of an example circulatory assist device in accordance with the present technology.

FIGS. 4A-4C are illustrative schematics of cross-sectional views of various example supports in a circulatory assist device, in accordance with the present technology.

FIG. 5A is an illustrative schematic of an example circulatory assist device, in accordance with the present technology.

FIG. 5B is an illustrative schematic of a radial inlet valve arrangement in an example circulatory assist device, in accordance with the present technology.

FIGS. 6A-6C are illustrative schematics of example vascular valves in a circulatory assist device, in accordance with the present technology.

FIG. 7A is an illustrative schematic of an example circulatory assist device, in accordance with the present technology.

FIG. 7B depicts an example circulatory assist device, in accordance with the present technology.

FIG. 8 is an illustrative schematic of an example circulatory assist device, in accordance with the present technology.

FIG. 9 is an illustrative schematic of an example circulatory assist device, in accordance with the present technology.

FIGS. 10A-10C are illustrative schematics of an example circulatory assist device in various states in a cardiovascular lumen, in accordance with the present technology.

FIG. 11A is an illustrative schematic of an example circulatory assist device, in accordance with the present technology.

FIG. 11B is an illustrative schematic of the example circulatory assist device shown in FIG. 11A placed in a descending aorta, in accordance with the present technology.

FIG. 12 is an illustrative schematic of an example circulatory assist device, in accordance with the present technology.

FIG. 13 is an illustrative schematic of an example circulatory assist device, in accordance with the present technology.

FIG. 14 is an illustrative schematic of an example circulatory assist device, in accordance with the present technology.

FIG. 15A is an illustrative schematic of a side view of an example circulatory assist device according to the present technology placed through a delivery sheath.

FIGS. 15B-15F are illustrative schematics of cross-sectional views of the circulatory assist device of FIG. 15A at various axial locations along the length thereof.

FIG. 16 is a schematic diagram of an example control unit of a circulatory assist device in accordance with the present technology.

FIGS. 17A-17C are illustrative schematics of various processes in methods for operating a circulatory assist device, in accordance with the present technology.

FIG. 18 is an illustrative schematic of an example circulatory assist device, in accordance with the present technology.

FIGS. 19A-19E are illustrative schematics of an example circulatory assist device during operation, in accordance with the present technology.

### DETAILED DESCRIPTION

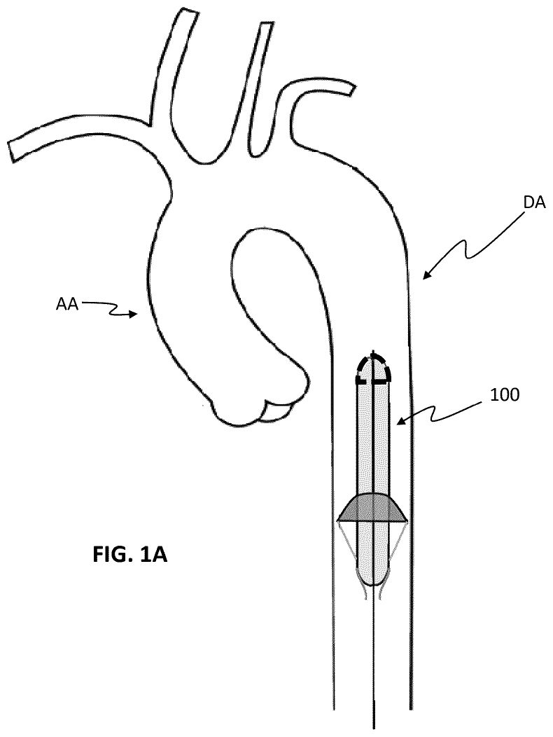

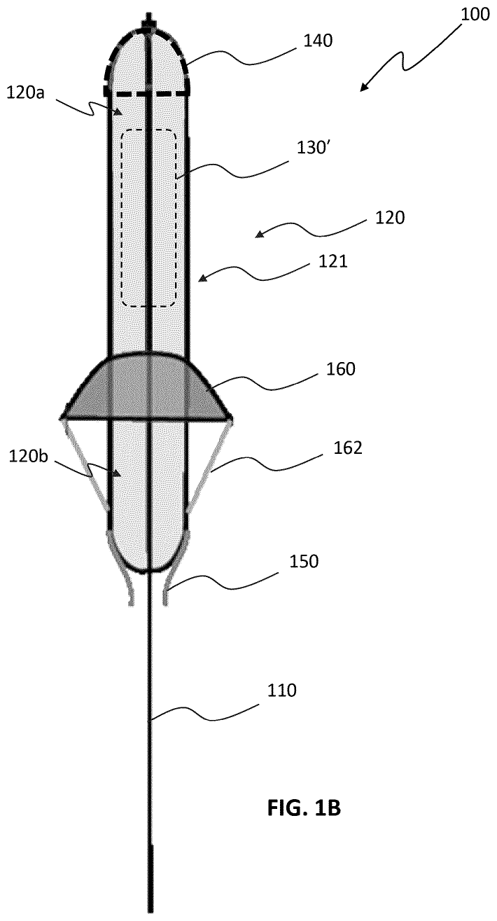

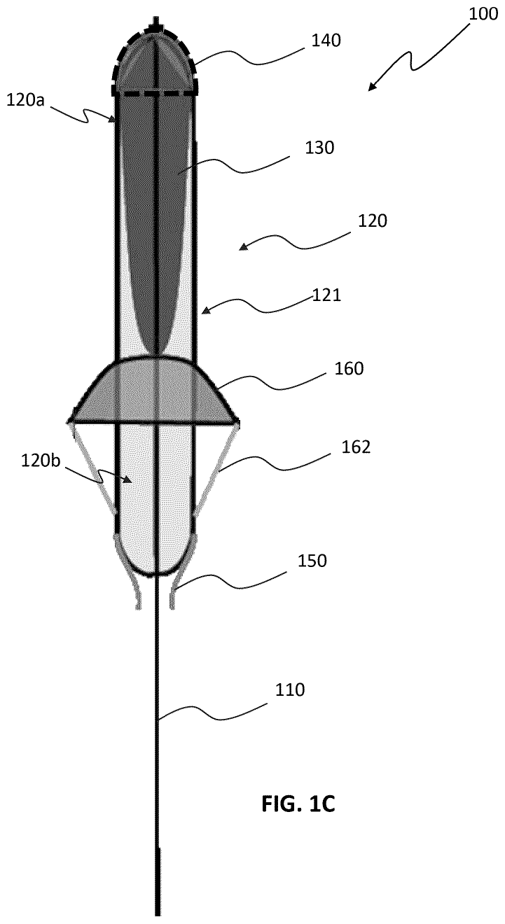

The present technology relates to circulatory assist systems and methods. Some aspects of the present technology, for example, are directed to cardiac assist devices and methods. Such devices can, for example, be delivered percutaneously into a cardiovascular lumen (also referred to herein as a “vascular lumen”) and are capable of pumping blood at flows high enough to support patients in cardiogenic shock, acute myocardial infarction, acute heart failure or during high-risk percutaneous coronary interventions, or other situations requiring hemodynamic support with reduced levels of hemolysis. Specific details of several aspects of the technology are described below with reference to FIGS. 1A-19E.

As used herein, the terms “proximal” and “distal” (and derivatives thereof) are used primarily within a frame of reference of a user placing a circulatory assist device within a patient, unless otherwise specified. For example, “proximal” primarily refers to a direction closer to the user, while “distal” primarily refers to a direction farther from the user. The term “upstream” in the vascular system means vascular locations closer to the patient's heart, while “downstream” means vascular locations further away from the patient's heart, regardless of the direction of blood flow through the vessel at the relevant time.

The circulatory assist devices and systems of the present technology may be used to provide circulatory assistance (e.g., cardiac assistance) in a variety of procedures and to address a variety of patient conditions. For example, the circulatory assist devices and systems may be used for cardiac assist during high-risk percutaneous coronary interventions (PCI) including angioplasty and stenting. Furthermore, the circulatory assist devices and systems may be used to provide cardiac support for patients experiencing cardiogenic shock. Furthermore, the circulatory assist devices and systems may be used to provide cardiac support for patients experiencing acute myocardial infarction. Generally, for such procedures, the circulatory assist devices will be configured for placement at least partially in the descending aorta. In some procedures, the circulatory assist devices can be additionally or alternatively configured for placement at least partially in the left ventricle. However, placement at various other cardiovascular lumen sites is also possible, including at least partially in the ascending aorta, the right atrium, right ventricle, and/or pulmonary artery.

### I. Circulatory Assist Systems

In some variations, a circulatory assist system includes a circulatory assist device (also referred to herein as a pump device) that is positionable in a patient (e.g., in a cardiovascular lumen, such as a blood vessel and/or heart chamber). For example, the circulatory assist device can function as a percutaneous ventricular assist device (pVADs), a transvalvular pVAD, or an intra-vascular and intra-ventricular blood pump, though other uses of the circulatory assist device are contemplated.

In some variations, a circulatory assist device may include a pump arrangement comprising a pump region configured to receive blood, an outflow region, and a pump (e.g., volume displacement member or impeller) in the pump region and configured to drive the received blood toward the outflow region. The circulatory assist device may further include a vascular seal (e.g., one-way valve, skirt structure) configured to form a peripheral seal between the pump arrangement and an inner wall of a cardiovascular lumen. Generally, in variations in which the pump arrangement includes a volume displacement member, the volume displacement member may be operable in an expansion phase and a contraction phase, such that cyclical operation between the expansion phase and the contraction phase pumps blood through the outflow region to provide circulatory assistance. In some examples, the vascular seal may be movable so as to form the peripheral seal during the pump expansion phase and to allow blood flow around the pump arrangement in the contraction phase. Similarly, in variations in which the pump arrangement includes an impeller pump, the impeller pump may be operable to propel blood to pump blood through the outflow region to provide circulatory assistance. In some examples, the vascular seal may be configured to open or close based on the pressure gradient across the seal, e.g., opening when the fluid pressure is higher upstream (closer to the heart) of the vascular seal, and closing when the fluid pressure is higher downstream (away from the heart) of the vascular seal. The vascular seal may alternatively be configured to remain continuously sealed against the vessel wall during pump operation.

In some variations, the circulatory assist device may be delivered to an aorta (e.g., descending aorta) of a patient. The option of aortic placement may be advantageous for a number of reasons. For example, placement of a circulatory assist device in the aorta is generally low invasive and/or results in low trauma, which may lower the risk of complications such as myocardial or cerebral infarction, and/or arrythmia that may result from interaction between the circulatory assist device and myocardium. As another example, placement of a circulatory assist device in the aorta reduces a length constraint of the device (e.g., compared to a circulatory assist device configured for placement at least partially across a cardiac valve), such that the circulatory assist device can be longer, thereby providing greater circulatory assistance over a longer anatomical distance (e.g., provide greater volumetric flow). As another example, placement of a circulatory assist device in the aorta may avoid interaction with the aortic valve, thereby reducing the potential for aortic valve injury that may result from a circulatory assist device being placed in a left ventricle of the patient. Furthermore, compared to placing a circulatory assist device in other cardiovascular regions such as the left ventricle, a procedure for placing a circulatory assist device in the aorta may be faster, simpler, and easier to perform. For example, the procedure may be simple enough for a clinician to perform using a skill level similar to that required to place an arterial line. As another example, the procedure to place a circulatory assist device in an aorta may be performed without imaging guidance, such that it can be performed in emergency situations (e.g., ambulance) in which X-ray or other imaging guidance may not be available. This may help contribute to faster patient treatment, as well as treatment with fewer complications.

As further described herein, in some variations the circulatory assist device may additionally or alternatively be placed in a cardiovascular lumen other than the aorta, and repositioned among two or more different cardiovascular locations to provide different levels of cardiovascular support (e.g., treatment intensity). For example, the circulatory assist device may be initially placed in the descending aorta to provide a first level of cardiovascular support, then repositioned to a transvalvular location in which the circulatory assist device is at least partially positioned in the left ventricle to provide a second level of cardiovascular support or treatment intensity greater than the first level of cardiovascular support or treatment intensity. When less cardiovascular support is needed by the patient, the circulatory assist device may then be repositioned again in the descending aorta to tune or adjust the provided cardiovascular support downwards, such as to wean the patient off cardiovascular support. However, in some variations the circulatory assist device may initially be at least partially placed in a left ventricle (and optionally, subsequently repositioned in the descending aorta to tune or adjust the provided cardiovascular support downwards, similar to that described above).

For similar reasons and for similar advantages, in some variations the circulatory assist device may be delivered to a pulmonary artery to provide support for a right ventricle of a patient. Additionally or alternatively, the circulatory assist device may be initially placed in the pulmonary artery to provide a first level of cardiovascular support, then repositioned to a transvalvular location in which the circulatory assist device is at least partially positioned in the right ventricle to provide a second level of cardiovascular support or treatment intensity greater than the first level of cardiovascular support of treatment intensity. When less cardiovascular support is needed by the patient, the circulatory assist device may then be repositioned again in the pulmonary artery to tune or adjust the provided cardiovascular support downwards, such as to wean the patient off cardiovascular support. However, in some variations the circulatory assist device may initially be at least partially placed in a right ventricle (and optionally, subsequently repositioned in the pulmonary artery to tune or adjust the provided cardiovascular support downwards, similar to that described above).

Another advantageous feature of a circulatory assist device in accordance with the present technology is the vascular seal (e.g., valve, skirt structure) forming a seal against a surrounding vascular lumen wall. In many instances, a conventional circulatory assist device placed in the aorta may have limited effectiveness in providing cardiovascular support, due at least in part by potential backflow or regurgitation around the device (e.g., from downstream to upstream back toward the aortic arch, etc.). In contrast, as further described herein, the vascular seal of the circulatory assist device in accordance with the present technology functions to help prevent backflow or regurgitation around the circulatory assist device to thereby improve the treatment effectiveness of the circulatory assist device.

For example, FIGS. 1A and 1B are schematic illustrations of an example variation of a circulatory assist device or blood pump device 100. As shown in FIG. 1A, the circulatory assist device 100 may be positioned in a descending aorta (DA) after advancing the circulatory assist device 100 from a percutaneous puncture site (e.g., insertion site at a femoral artery). However, in other variations the circulatory assist device 100 may be positioned in another vascular lumen (e.g., pulmonary artery). As shown in FIG. 1B, the circulatory assist device 100 may include a pump arrangement 120 including a pump region 120a configured to receive blood, an outflow region 120b, and a pump 130′ in the pump region and configured to drive the received blood toward the outflow region 120b. In some variations, the circulatory assist device 100 may further include a vascular seal 160 configured to form a peripheral seal between the pump arrangement 120 and an inner wall of the aorta (or other cardiovascular lumen such as a pulmonary artery).

In some variations, the pump arrangement 120 can include a pump body 121 configured to receive a fluid (e.g., blood) when placed in a cardiovascular lumen of a patient and convey the fluid back to the cardiovascular lumen for circulatory assistance. The pump body 121 can include a conduit with a pump region 120a and an outflow region 120b, where the pump region 120a is more distal than the outflow region 120b. Generally, the pump body 121 (at least the pump region 120a, for example) may have an elongated shape, such as a tubular or pipe-like shape. The pump body 121 may, for example, have at least a portion that is cylindrical or elongated with an elliptical cross-section. However, in some variations the pump body 121 may have an elongated shape with a varying cross-section (e.g., the pump body 121 may be bulbous or hourglass-shaped) along the conduit length.

As further described below, the pump body 121 may further include at least one inlet valve 140 configured to receive a fluid through the inlet of the conduit along the flow axis, and a pump 130′ arranged in the conduit. The pump 130′ may, for example, include a volume displacement member 130 (as shown in FIG. 1C, for example), impeller, or other suitable pump mechanism. The volume displacement member 130, if present, may be operable in an expansion phase and a contraction phase. For example, in some variations the volume displacement member 130 may include a balloon, and the balloon may be inflated in the expansion phase, and deflated in the contraction phase (e.g., at a high frequency as described elsewhere herein). Furthermore, in some variations, the pump body 121 may or may not further include at least one one-way outlet valve 150 configured to allow fluid flow from the outflow region 120b, away from the pump arrangement 120, and into the cardiovascular lumen, but block fluid flow from the cardiovascular lumen into the pump body 121. As another example, the pump 130′ may include an impeller which operates in a continuous rotational manner to propel blood. In such variations where the pump 130′ includes an impeller, the pump body 121 may omit both an inlet valve 140 and an outlet valve 150, and the impeller may be operated without rapid pulsation between an expansion phase and a contraction phase as described herein with respect to a volume displacement member 130.

In some variations, the pump body 121 may be coupled to or otherwise arranged on a catheter 110, which can be used to position the pump body 121 in the patient and/or facilitate operation of the volume displacement member 130 (if present) in the expansion phase and the contraction phase. For example, the pump body 121 may be coupled to a distal portion of the catheter 110, while a proximal portion (not shown in FIG. 1A) of the catheter 110 may be outside the patient and coupled to a control system and/or actuator for controlling the volume displacement member 130 in the expansion phase and the contraction phase. In some variations, the catheter 110 may include other control elements for the pump (e.g., power connection for an impeller or other pump device).

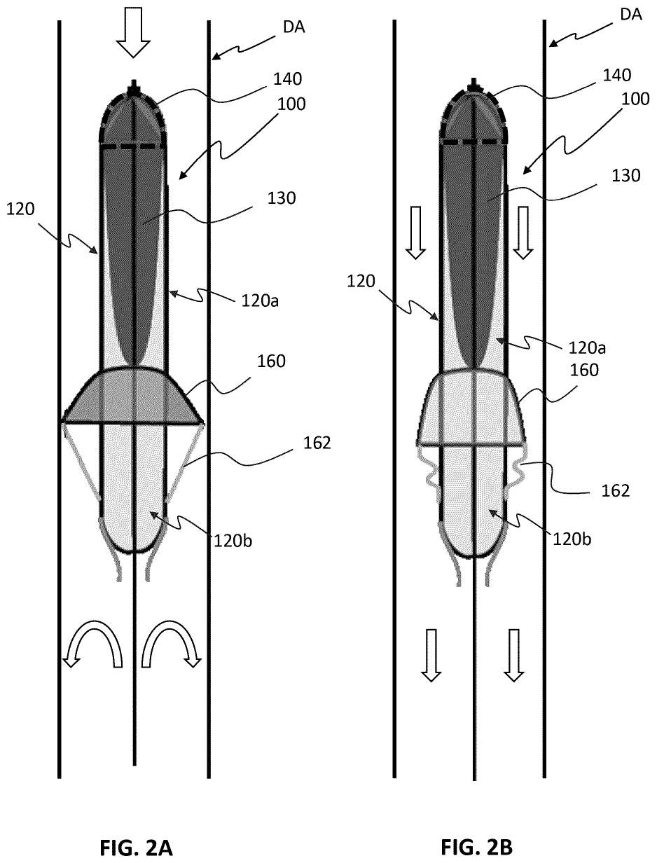

FIGS. 2A and 2B are schematic illustrations of an example circulatory assist device 100 during use. The circulatory assist device 100 may include a volume displacement member 130. In the example device shown in FIGS. 2A and 2B, the volume displacement member 130 includes a balloon that is cyclically inflated and deflated within the pump body 121. Native flow of fluid (e.g., blood) that enters the pump arrangement 120, such as through the inlet valve 140, can be driven (e.g., accelerated) toward the outflow region 120b when expansion of the balloon causes displacement of the blood. Generally, the exit of accelerated fluid flow from the pump arrangement 120 may help entrain the native flow fluid via transfer of fluid momentum, thereby resulting in a net increase in overall volumetric flow in the cardiovascular lumen in which the circulatory assist device is placed. This accelerated fluid flow from the pump arrangement 120 may also be achieved in variations in which the pump arrangement 120 includes other kinds of pump devices, such as an impeller pump.

As shown in FIG. 2A, when the circulatory assist device 10 is in an active state providing circulatory support, the vascular seal 160 is in a closed state in which it forms a peripheral seal against the inner wall of the descending aorta (DA), thereby substantially preventing fluid from passing through the descending aorta across the vascular seal 160 in either direction. In particular, as shown in FIG. 2A, when the vascular seal 160 is in this closed state, the vascular seal 160 substantially prevents backflow of fluid in a proximal-to-distal direction. Various examples of vascular seals are described in further detail herein.

In some variations, the vascular seal 160 may be in this closed state when pressure on a proximal (downstream) side of the vascular seal is greater than the distal (upstream) side of the vascular seal. In some variations as further described herein, the vascular seal 160 may also be in this closed state when pressure on the proximal (downstream) side of the vascular seal is about equal to the distal (upstream) side of the vascular seal. Closure of the vascular seal 160 may help to maintain a pressure gradient in the descending aorta that is created by the circulatory assist device. For example, when the vascular seal 160 is closed and the circulatory assist device 100 is in the active state as shown in FIG. 2A, the vascular seal 160 maintains a higher pressure in the proximal side of the vascular seal 160 and in the peripheral system, thereby maintaining organ perfusion, especially kidney perfusion. This arrangement also helps to maintain a lower pressure on the distal side of the vascular seal 160, thereby advantageously reducing the workload for the heart.

Furthermore, in some variations, the vascular seal may be configured to gradually shape the outflow of fluid from the pump arrangement 120 from a narrower, more focal profile to a wider, more divergent profile. This widening or diverging of the outflow of fluid may help reduce the exit speed of the pumped fluid, thereby reducing the likelihood of turbulent flow that may cause complications such as hemolysis.

FIG. 2B illustrates the circulatory assist device 100 in a passive state, in which the vascular seal 160 is in an open state and does not form a peripheral seal between the pump arrangement 120 and the inner wall of the descending aorta. The vascular seal 160 may be in this open state when pressure on the distal (upstream) side of the vascular seal is greater than pressure on the proximal (downstream) side of the vascular seal. Additionally or alternatively, the vascular seal 160 may transition to the open state through activation of one or more actuators (e.g. proximal movement of pull wires, as further described below). In some variations, when the circulatory assist device 100 is in the passive state, fluid is allowed to flow (e.g., through entrainment) through the descending aorta while bypassing the pump arrangement 120. However, in some variations, when the circulatory assist device 100 is in the passive state, fluid is allowed to flow (e.g., through entrainment) through the descending aorta around the pump arrangement 120, while also flowing through the pump arrangement 120. In this passive state, the volume displacement member 130 may be still cyclically inflated and deflated within the pump body 121, or may remain static without such cyclical inflation and deflation.

Further details of various features and variations of the circulatory assist device and methods of treatment using the circulatory assist device are described below.

### A. Pump Arrangement

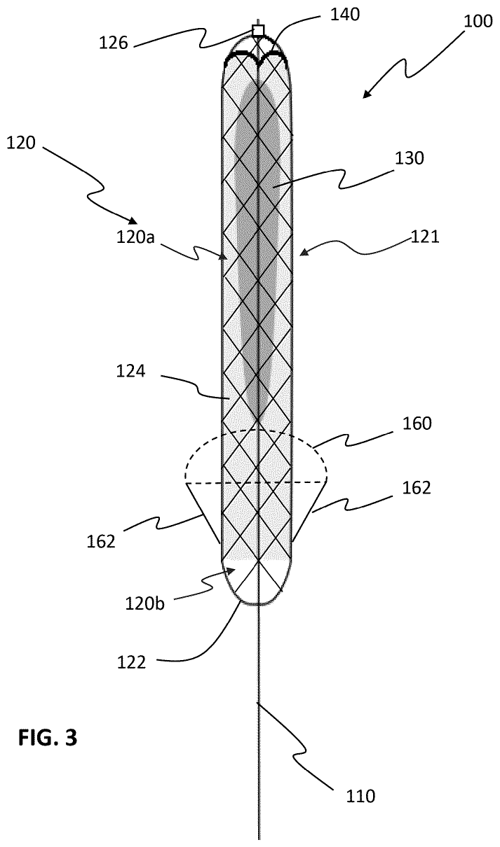

As described above, the circulatory assist device 100 may include a pump arrangement 120 that functions to receive a fluid (e.g., blood) when placed in a cardiovascular lumen and pump the fluid to provide circulatory assistance. FIG. 3 is another schematic illustration of a circulatory assist device 100 including a more detailed view of the pump arrangement 120, with the vascular seal 160 depicted in outline form in broken line for reference.

In some variations, the pump arrangement 120 may include a pump body that is flexible (or pre-formed with a suitable contour or other shape) to conform to surrounding anatomy and avoid tissue trauma. As shown in FIG. 3 for example, the pump body 121 may include a conduit with an expandable support 122 and at least one fluid impermeable membrane 124 adjacent to the expandable support. The pump body, for example by virtue of the geometric and/or material properties of the support 122 and/or the membrane 124, may be substantially non-compliant so as to resist deformation during the expansion and contraction cycles of the volume displacement member 130. Furthermore, the conduit may have a circular, ellipsoidal, or other suitable curved wall, so as to help the conduit (and the overall pump body) be more resistant to failure in response to internal pressure (i.e., positive and/or negative pressure). Additionally, the curved wall of the conduit may help facilitate suitable clearance between the outer surface of the volume displacement member 130 and the conduit, as further described herein.

The support 122 functions at least in part to provide structural support to the pump body. For example, the support 122 may help the conduit to be resistant against diametrical expansion in response to increased pressure when filled with blood and during expansion of the volume displacement member. Such non-distensibility allows spacing to be maintained between the pump body and the vessel (e.g., aortic, pulmonary-arterial or ventricular) wall to minimize trauma to cardiovascular tissue and also increases pump efficiency. Additionally, the support 122 may help the conduit be resistant against collapsing in response to decreased pressure (e.g., during contraction of the volume displacement member). In some variations, the support 122 may be configured to be collapsible or crimpable into a lower profile transport state during delivery to the target placement location (e.g., descending aorta or left ventricle), and/or when subject to sufficient external forces to allow for endovascular delivery and retrieval. The support 122 may further be configured to expand into a deployed state, such as by self-expansion and/or expansion with another device (e.g., balloon-expandable). In some variations, the support 122 may include a frame or skeleton of a resilient metal such as nickel-titanium alloy, cobalt-chrome, chromoly steel, or stainless steel, etc., or a suitable polymeric material such as nylon. The support 122 may, for example, include woven wires, mesh, a basket, laser-cut material, or a monolithic tube having an arrangement of openings, slits, or cells which allow expansion in at least one dimension from the transport state to the deployed state. For example, the support 122 can include a plurality of struts or cells arranged in a radially-expandable geometry. The support 122 can include a single continuous body, or can include multiple bodies coupled together (e.g., nested mesh tube structures with overlapping walls).

In some variations, at least a portion of the support 122 may have a generally tubular shape. For example, at least the portion of the support 122 forming the pump region 120a can be tubular. In some variations, at least a portion of the support 122, such as at least the portion of the support 122 forming the pump region 120a, can be tubular with a constant cross-sectional shape (e.g., cylindrical) or a varying cross-sectional shape along its length (e.g., bulbous, hourglass-shaped). The support 122 can have at least one closed end. For example, as shown in FIG. 3, in some variations the distal end of a generally tubular support 122 may be closed by a connector 126 (e.g., crimp, tube, etc. that may connect free ends of wires forming the support 122) or other suitable mechanical fastener(s), and/or joined in any other suitable manner such as welding, etc. Furthermore, in some variations the proximal end of the support 122 (e.g., the proximal end of the outflow region 120b) may be closed through integral formation, such as by virtue of the pattern of weaving, mesh, laser-cutting, etc. It should be understood that in some variations, either or both of the distal end of the support 122 and the proximal end of the support 122 may additionally or alternatively be closed through integral formation, mechanical fastener(s), welding, and/or the like. Furthermore, in some variations, the distal end of the support 122 and/or the proximal end of the support 122 may instead be open.

In some variations, the pump body may include one or more features to aid repositioning or retrieval of the pump body from the patient (e.g., after circulatory assistance is no longer needed, or if the pump body is to be swapped with another circulatory assist device). For example, a pigtail connector may be coupled to or integrally formed with an end of the pump body, such as coupled to or integrally formed with the support 122.

In some variations, the pump body 121 of the pump arrangement 120 may include at least one fluid impermeable membrane 124 adjacent to a surface of the support 122. The membrane 124 may extend along at least a portion of the length of pump body. For example, as shown in FIG. 3, the membrane 124 may extend along at least a portion of the pump region 120a of the pump body. The membrane 124 may further extend along at least a portion of the outflow region 120b. In some variations, at least a portion of the outflow region 120b may remain uncovered by the membrane 124 so as to permit passage of fluid through the support 122 in and/or out of the conduit (e.g., through open and uncovered cells of the support 122). The membrane 124 may include one continuous layer of material, or may include multiple segments of material that are coupled to one another (e.g., radial or longitudinal strips sealed to one another, such as by heat welding, electrospinning, etc.).

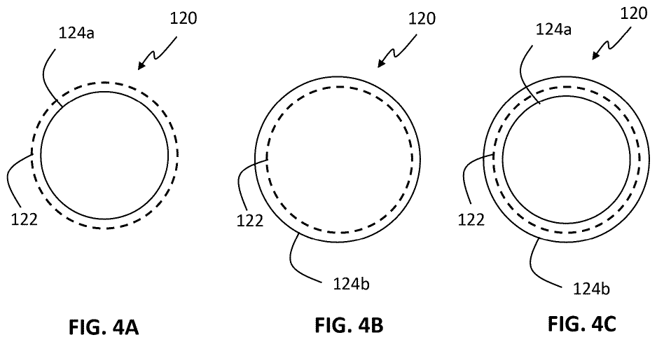

The pump body 121 of the pump arrangement 120 may include at least one membrane 124 adjacent to an inner surface and/or an outer surface of the support 122. In some variations, one or more membranes 124 may be adjacent to an inner surface and/or an outer surface of the support 122. That is, the one or more membranes 124 may include an inner membrane 124a and/or an outer membrane 124b. For example, as shown in the cross-sectional view depicted in FIG. 4A, the pump body may include an inner membrane 124a coupled to an inner surface of the support 122.

As another example, as shown in the cross-sectional view depicted in FIG. 4B, the pump body may include an outer membrane 124b adjacent to an outer surface of the support 122. In this example of FIG. 4B, the outer membrane 124b may be coupled to the outer surface of the support 122, or may be unattached from the outer surface of the support 122. In variations in which the outer membrane 124b is not attached to the outer surface of the support 122, the outer membrane 124b may overlie the outer surface of the support 122 such that the outer membrane 124b is configured to expand in tandem with the support 122 when the support 122 is expanded. In these variations similar to the example of FIG. 4B, the outer membrane 124b may help contain radially outward pressure in the pump body, while the support 122 may help prevent collapse of the pump body by providing outward support against negative (e.g., inward) pressure.

As yet another example, as shown in the cross-sectional view depicted in FIG. 4C, the pump body may include both an inner membrane 124a coupled to an inner surface of the support 122, and an outer membrane 124b coupled to an outer surface of the support 122, such that at least a portion of the support 122 is sandwiched between membrane layers. Furthermore, the pump body may include more than one layer of an inner membrane 124a (e.g., two or more inner membrane layers), and/or more than one layer of an outer membrane 124b (e.g., two or more outer membrane layers). In variations in which the pump body includes both an inner membrane 124a and an outer membrane 124b, the inner membrane 124a and the outer membrane 124b may extend over the same portions of the support 122, or may extend over different portions of the support 122.

The inner membrane 124a and/or the outer membrane 124b may be coupled to the support in any suitable manner, including, for example, spray lamination, welding, bonding, and/or adhesive. Furthermore, in some variations, the support 122 may be at least partially embedded within a fluid impermeable membrane 124 (e.g., via overmolding or other suitable technique). In some variations, the one or more membranes 124 may be coupled to the support 122 continuously along the inner and/or outer surfaces of the support 122. However, in some variations, at least some of the one or more membranes 124 may be coupled to the support 122 at only a portion of the inner and/or outer surfaces of the support 122, such as only along certain selected axial locations of the support 122 and/or certain selected radial locations around the support 122).

In some variations, the material of the one or more membranes 124 may be flexible and durable, such as nylon or polyurethane with high durometer values. This can be achieved by using a polymer with high tensile modulus. For example, the membrane comprises a TPU such as pellathane or tecothane. In one example, the membrane can include tecothane in a durometer of approximately 72D, which may accommodate the stress placed on the conduit during operation of the circulatory assist device 100, without undergoing plastic deformation. In some variations, the one or more membranes 124 may include an inelastic (e.g., non-compliant) material. For example, an inelastic material for the one or more membranes 124 may be suitable in variations in which the membrane(s) 124 are coupled to the support 122 at only a portion of the inner and/or outer surfaces of the support 122.

In some variations, the pump body may also include one or more circumferential fibers of a material with a high tensile strength, which function to further limit the distensibility of the support 122 beyond its desired size, while still allowing the pump body to be radially collapsed into a transport state (e.g., for insertion and removal). Such circumferential fibers may be arranged, for example, circumferentially around at various axial locations along the pump region 120a of the pump body. The circumferential fibers can include any suitable material such as Kevlar, spectra, carbon nanotubes, and/or other such materials that are attached, embedded within, or woven into the support 122 and/or membrane 124.

As described above, in some variations the pump body of the pump arrangement 120 may further include at least one inlet valve 140 configured to convey fluid into the pump body. The inlet valve 140 may be a one-way valve with a preferential flow direction, where the one-way valve permits flow into the pump body 121 through the inlet valve, while substantially preventing flow out of the pump body 121 through the inlet valve. For example, the inlet valve 140 may include a duckbill valve, or a valve with multiple leaflets (e.g., bicuspid valve, tricuspid valve, etc.). In some variations, the inlet valve 140 may be a passive valve configured to open and close in response to pressure change, though in some variations the inlet valve 140 may additionally or alternatively be an active valve whose opening and closure may be controlled by a suitable actuator.

The inlet valve 140 may be configured to withstand opening and closing at a high frequency over a sustained period of time (e.g., high fatigue resistance). For example, in some variations, the inlet valve 140 may be configured to have a fast closure response time, such as about 4 milliseconds or less, about 3 milliseconds or less, about 2 milliseconds or less, about 1.5 milliseconds or less, or about 1 millisecond or less.

In some variations, the inlet valve 140 may be arranged to permit axial flow of fluid into the conduit. In these variations, an axial orientation of the inlet valve 140 may be advantageous for reducing the diameter of the pump body 121 in the collapsed (e.g., crimped) configuration. For example, as described above, the inlet valve 140 may be arranged at an axial location between the pump 130′ and the inlet of the pump body 121, such that the inlet valve 140 does not overlie the pump 130′ and thus does not add additional radial bulk to the profile of the pump body 121 in the collapsed configuration. Furthermore, in some variations the axial inlet valve 140 may itself be low-profile, thereby further contributing to a smaller profile of the pump body 121 in the collapsed configuration.

Although FIGS. 1A-3 depict a circulatory assist device 100 including one inlet valve 140, it should be understood that in some variations, the circulatory assist device 100 may include multiple (e.g., two or more) inlet valves.

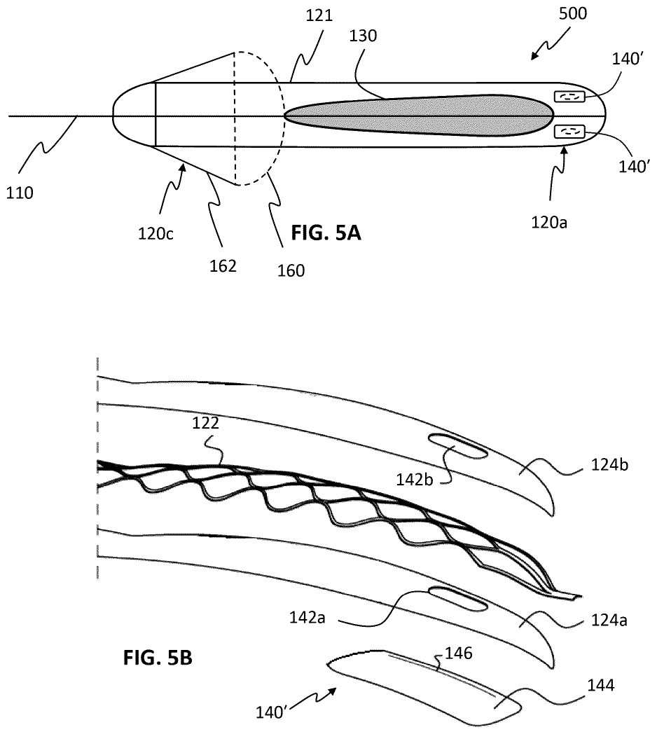

Furthermore, in some variations, in addition to or as an alternative to an inlet valve receiving fluid in an axial direction, the circulatory assist device 100 may include one or more inlet valves configured to receive fluid in a direction not aligned with the flow axis of the conduit. For example, as shown in FIG. 5A, in some variations, a circulatory assist device 500 may be similar to the circulatory assist device 100, except that the circulatory assist device 500 includes one or more inlet valves 140′ configured to receive fluid in a radial direction. The inlet valves 140′ may, for example, be a flap valve configured as a one-way valve, and may include an aperture formed in a wall of the conduit of the pump body, and a flap (e.g., coupled to or integrally formed with the membrane 124). The flap may be configured to alternate between exposing the aperture (thereby permitting fluid flow into the conduit) and covering the aperture (thereby substantially preventing fluid flow from exiting the conduit). An example variation of an inlet flap valve 140′ is shown in FIG. 4B. As shown in FIG. 5B, an inner membrane 124a may be arranged on an inner surface of the support 122, and an outer membrane 124b may be arranged on an outer surface of the support 122. The inner membrane 124a and the outer membrane 124b may have respective apertures 142a and 142b that at least partially overlie each other (and at least partially overlie an opening of the support 122). A flap 144, which may be attached to the inner membrane 124a at a connection 146 on one side of the flap 144 (or alternatively may be integrally formed with the inner membrane 124a, such as cut out from the inner membrane 124a) is configured to overlie the apertures 142a and 142b. Pressure differential between inside and outside the pump body 121 may cause movement of the flap 144 relative to the apertures 142a and 142b. For example, movement of the flap 144 away from the inner membrane 124a may allow fluid to enter the conduit in a radial direction. Conversely, movement of the flap 144 toward the inner membrane 124a may substantially prevent fluid from exiting the conduit. Although FIG. 5B illustrates one variation of an inlet flap valve 140′, other variations of flap valves (and other kinds of radial valves) may be constructed in accordance with the present technology. For example, the pump body may include only an inner membrane 124a with at least one aperture 142a, or only an outer membrane 124b with at least one aperture 142b. Additionally or alternatively, the flap 144 may be arranged internal to the support 122, or external to the support 122. Other variations of suitable flap valves are described in greater detail in International Patent Application No. PCT/EP2023/059293, which is incorporated herein in its entirety by this reference.

Furthermore, in some variations, the circulatory assist device 100 may include one or more outlet valves arranged at or near the outflow region 120b of the pump body. The outlet valve(s) may, for example, help regulate the exit of fluid from the pump body by substantially occluding fluid flow out of the pump body during certain phase(s) of operation. However, in some variations the circulatory assist device 100 may omit outlet valves arranged at or near the outflow region 120b of the pump body, as shown in FIG. 3.

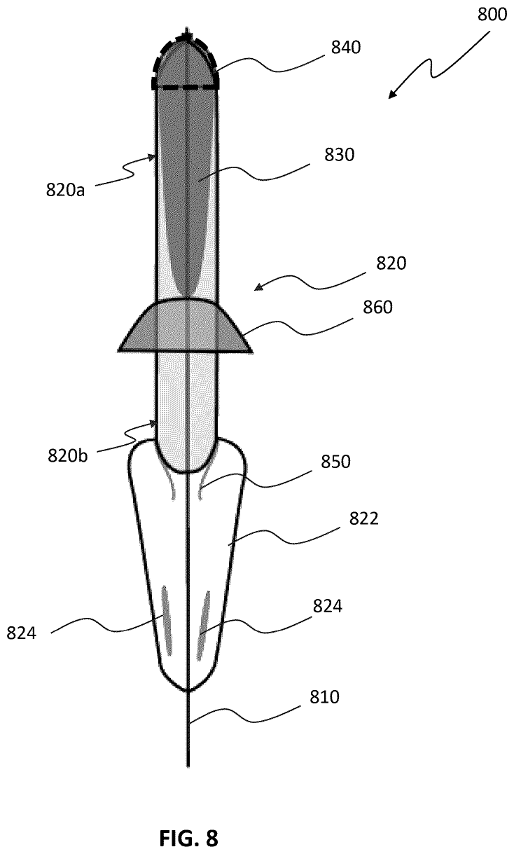

In some variations, the pump arrangement may include an outflow structure coupled to a proximal region of the pump body, such that the outflow region may be arranged at least partially in the outflow structure. In some variations, the outflow structure includes a flexible or compliant chamber with one or more outlets. Due at least in part to the compliance of the chamber, the outflow structure may be configured to help buffer the fluid exiting the pump body and reduce turbulence of such outflow of fluid, thereby reducing the likelihood of complications such as hemolysis. In some variations, the outflow structure may include a flexible material such as a flexible membrane (e.g., the same or similar material as the membrane 124 of the pump body). In some variations, the chamber material can be an extension of the membrane 124 of the pump body, while in some variations the chamber material can be formed separately and coupled (e.g., thermally joined) to the pump body. FIG. 8 illustrates an example circulatory assist device 800 including a pump arrangement 820 that is similar to the pump arrangement 120 described herein with similar numbering of features, except the pump arrangement 820 includes an outflow structure 822 that is in fluidic communication with a pump body including the volume displacement member 830. The outflow structure 820 may include a flexible or compliant chamber that receives fluid from the pump body of the pump arrangement 820, and conveys pumped fluid out of the pump arrangement 820 via one or more outlets 824. The outlets 824 may include one or more outlet valves (e.g., flap valves, such as valves similar to inlet flap valves 140′ described above with respect to FIGS. 5A and 5B).

As described herein, the circulatory assist device 100 may include at least one pump 130′. For example, the circulatory assist device 100 may include a volume displacement member 130. The volume displacement member 130 functions to urge or otherwise drive fluid (e.g., blood) through the outflow region 120b of the pump arrangement 120 to provide circulatory assistance, after such fluid has been received in the pump region 120a of the pump arrangement 120. In some variations, the volume displacement member 130 may be arranged in the pump region 120a of the pump region 120. Although operation of the pump 130′ is primarily described herein with reference to operation of a volume displacement member 130, it should be understood that many aspects (e.g., fluid flow requirements, positioning of the pump relative to other components of the circulatory assist device 100, etc.) are equally applicable to other kinds of pumps, including but not limited to impeller pumps.

The volume displacement member 130 may include any of various types of mechanisms capable of displacing a volume of fluid in a cyclical, repeating manner. In some variations, the volume displacement member 130 may include an inflatable balloon that can be inflated with a fluid to an expanded, high-volume state and deflated partially or completely to a contracted, low-volume state. In other variations, a piston, bellows, accordion-style expandable body, and/or other type of volume displacement member may be used. The volume displacement member is capable of moving cyclically between the contracted low-volume state in which it occupies a smaller portion of the conduit, to an expanded high-volume state, in which it occupies a substantially larger portion of the conduit, thus displacing blood therefrom. The volume displacement member 130 may be configured to cyclically move between these contracted and expanded states at a high frequency, such as at least about 300 beats per minute, at least about 500 beats per minute, at least about 1000 beats per minute, or at least about 1200 beats per minute, at least about 1500 beats per minute, at least about 2000 beats per minute, at least about 2500 beats per minute, at least about 3000 beats per minute (e.g., between about 1000 beats per minute and about 3000 beats per minute). In some variations, the frequency of the contraction/expansion cycle of the volume displacement member 130, in combination with the features (e.g., dimensions) of the rest of the pump body, is controlled such that the pump body is configured to convey fluid through the outflow region (and out the pump arrangement 120 via one or more outlets) with a flow rate of at least about 5 L/min.

When in the fully expanded state, the volume displacement member 130 may have a maximum diameter that is smaller than the inner diameter of the support 122, thereby allowing the outer surface of the expanded volume displacement member 130 to be spaced apart from the support 122, which provides clearance for fluid to move through the conduit between the inlet and the outlet even when the volume displacement member 130 is fully expanded. Such clearance may, in some instances, further function to help limit hemolysis during high frequency operation of the volume displacement member 130. For example, in some variations, when the volume displacement member 130 is fully expanded, a spacing of at least about 0.05 mm, or at least about 1.0 mm-5.0 mm (e.g., about 1.0 mm-3.0 mm) may be maintained between the volume displacement member 130 and an interior surface of the support 120.

As further described below with respect to the catheter 110, the volume displacement member 130 may be coupled to a shaft of the catheter 110. Additionally or alternatively, in some variations, the volume displacement member 130 may be coupled to the conduit of the pump body (e.g., support 122 and/or membrane 124), which may help to anchor the volume displacement member 130 in a fixed position relative to the pump body, thereby minimizing movement of the volume displacement member 130 relative to the pump body (other than from inflation) and reducing vibration of pump body.

In variations in which the volume displacement member 130 is a balloon, it may include a durable material such as polyurethane or nylon. The balloon may be formed of a single, thin wall of such material. For example, in one illustrative variation, the balloon may be made of pellethane 55D (or a material with similar mechanical properties), and may have a wall thickness of about 10 μm-60 μm (e.g., about 20 μm). As shown in FIG. 1B and FIG. 3 for example, the balloon may have a generally elongated shape and extend longitudinally along a portion of the length of the pump body. For example, the balloon in its expanded state may have a generally ellipsoidal shape.

### B. Vascular Seal

In some variations, the circulatory assist device 100 may further include a vascular seal 160 configured to form a peripheral seal between the pump arrangement 120 and an inner wall of a cardiovascular lumen (e.g., aorta, such as the descending aorta). As described above, the vascular seal 160 may have a closed state in which the vascular seal forms the peripheral seal between the pump arrangement 120 and the inner wall of a cardiovascular lumen, and an open state in which the vascular seal does not form such a peripheral seal, allowing blood to flow around the pump arrangement 120. When the circulatory assist device 100 is deployed at a treatment site, the vascular seal 160 may toggle between its closed state and open state at least in part passively (e.g., in response to pressure differential across the vascular seal 160), and/or may toggle between its closed state and open state at least in part actively in response to actuation (e.g., pull wires, inflatable or otherwise expandable members, self-expansion, etc.). As further described above, when closed, the vascular seal 160 may advantageously help reduce backflow or regurgitation of blood around the circulatory assist device 100, help maintain a pressure gradient in the descending aorta that is created by the circulatory assist device and/or help shape the outflow of fluid to have a wider, more divergent profile that reduces exit speed of the fluid and reduces the likelihood of hemolysis and/or other complications.

In some variations, the vascular seal may engage with the cardiovascular lumen in an atraumatic manner (e.g., without anchoring). Varying pressure gradients within the cardiovascular lumen during device operation may urge the circulatory assist device 100 to move (e.g., longitudinally, rotationally, etc.), so engagement with the cardiovascular lumen may be advantageous in some instances to help avoid trauma to surrounding tissue. Accordingly, the vascular seal may include a soft, flexible material with a smooth peripheral edge and smooth valvular surface(s). Furthermore, the vascular seal may omit anchoring features (e.g., tines, hooks, etc.) along the peripheral edge and/or on the valvular surface.

In some variations, the vascular seal may be configured to radially expand (e.g., from a first diameter to a second diameter larger than the first diameter) to form the peripheral seal against the inner wall of a cardiovascular lumen. For example, the vascular seal may be configured to radially expand into the closed state of the vascular seal. In some variations, this radial expansion may involve self-expansion of the vascular seal. For example, the vascular seal may include shape memory material (e.g., nitinol) configured to form the peripheral seal when released from constraint of an outer sheath (not shown). As such, the vascular seal may in some instances be biased toward the closed state (e.g., to help build the pressure gradient in the cardiovascular lumen in a rapid, autonomous manner). However, in some variations, the radial expansion of the vascular seal may additionally or alternatively be actively actuated. For example, the vascular seal may be at least partially inflatable (e.g., fillable with an inflation fluid to radially expand), and/or may be expanded with a separate mechanism such as a balloon.

Furthermore, in some variations the vascular seal may be radially collapsible to a delivery state, so as to help facilitate a low profile of the circulatory assist device 100 during device repositioning and/or retrieval. For example, the delivery state of the vascular seal may be the same as the closed state of the vascular seal described herein, or the delivery state may be different (e.g., a more radially contracted or collapsed form of the vascular seal compared to the closed state of the vascular seal).

In some variations, the vascular seal may be actively radially collapsed to the delivery state with one or more actuators. For example, as shown in FIG. 1B, one or more tethers 162 may be coupled to the vascular seal at or near a peripheral portion of the vascular seal, and extend proximally along the catheter 110. The vascular seal may be radially collapsed to a delivery state by proximally pulling on the tethers 162, thereby drawing the peripheral portion of the vascular seal inwards to collapse or narrow the vascular seal and/or elongating the vascular seal. Additionally or alternatively, the tethers 162 may limit the radial expansion of vascular seal 160 when moving to the open position so as to optimally seal with the vascular lumen. Tethers 162 may further function to help prevent the vascular seal from inverting (e.g., flipping inside out) under fluid pressure, thereby allowing fluid flow past the vascular seal. However, in some variations the vascular seal may be actuated or otherwise permitted to invert (e.g., inversion of a membrane, flap, etc.) to transition from the open state to the closed state. In some variations, the circulatory assist device 100 may include multiple tethers 162 arranged equally or unequally circumferentially around the vascular seal. Examples of tethers 162 include wires (e.g., nitinol wires), sutures, cord, flexible strips (e.g., polymer strips), and/or other suitable attachment member(s). For example, in some variations, the tethers 162 may be integrally formed with the support 122 of the pump arrangement 120, such as wire extensions of a mesh forming the support 122 of the pump arrangement 120.

In some variations, the vascular seal may be configured to form a seal against the inner wall of cardiovascular lumens (e.g., aorta) of varying anatomical sizes and/or shapes. For example, the vascular seal 160 may comprise a flexible membrane or skirt configured to seal (e.g., appose) against varying lumen diameters, by including a flexible structure that is expandable to a range of varying valve diameters and/or has a tapered profile along which the vascular seal has varying diameter. In some variations, the vascular seal 160 may have a generally outwardly flaring shape (e.g., dome, umbrella, skirt, cone, cup, and/or the like), with a central closed upstream or distal end and a peripherally open downstream or proximal end. Additionally or alternatively, the vascular seal may include a plurality of segments (e.g., discrete segments, folded segments that are connected such through corrugation and/or pleats) that may move relative to one another to conform to the profile of the inner wall of a cardiovascular lumen.

In some variations, the vascular seal may include a flexible membrane substantially impermeable to fluid. The membrane may, for example, be similar to the membrane 124 of the pump arrangement 120. The membrane of the vascular seal may be integrally formed with the membrane 124 (e.g., as an extension of the membrane 124), or may be separately formed and joined to the pump arrangement 120 at a suitable location (e.g., through thermal welding, etc.). In some variations, the membrane may be supported by one or more reinforcement members to help support the shape of the vascular seal 160. The reinforcement members may, for example, include wire (e.g., nitinol), and/or one or more inflatable reinforcement members. In some variations, one of more of the reinforcement members may be integrally formed with the support 122 of the pump arrangement 120, such as wire extensions of a mesh forming the support 122 of the pump arrangement. Similar to that described above with respect to FIGS. 4A-4C for a pump body 121 of the pump arrangement 120, in some variations the membrane 124 can include an inner membrane and/or an outer membrane, each of which is adjacent to an inner side and/or outer side of the reinforcement membranes.

However, in some variations the membrane may be a material sufficiently rigid on its own (without supplemental reinforcement) to maintain a desired shape of the vascular seal 160 while still being flexible and compliant enough to transition between closed and open states.

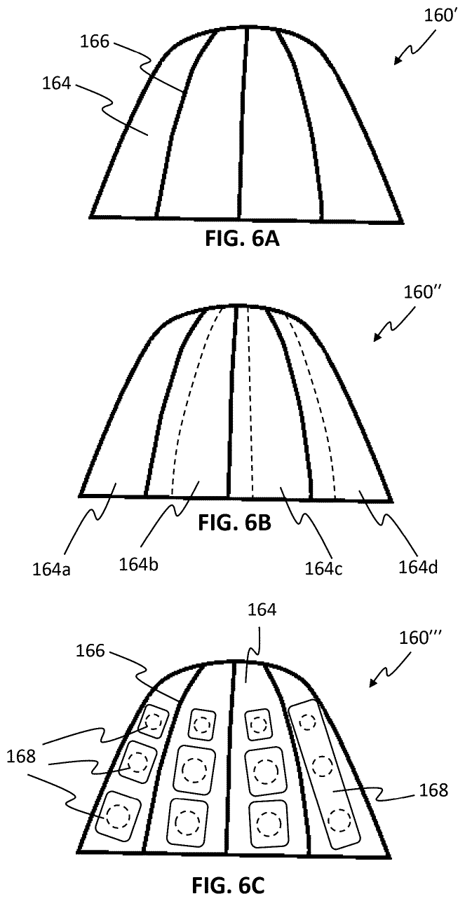

Various examples of architectures for the vascular seal 160 are shown in FIGS. 6A-6C. FIG. 6A is a schematic illustration of an example vascular seal 160′ including a membrane 164 and a plurality of reinforcement members 166. The reinforcement members 166 may be radially distributed (e.g., similar to umbrella arms) to form a support structure for the membrane 164. In some variations, the membrane 164 may include a sheet of material extending peripherally around the vascular seal 160a. Additionally or alternatively, the membrane 164 may include multiple radial segments of material interspersed between adjacent reinforcement members 166 (and may be coupled to one or more reinforcement members 166, such as by suturing).

FIG. 6B is a schematic illustration of an example vascular seal 160″ including multiple leaflets or segments of membrane material arranged in an overlapping manner. For example, at least four leaflets 164a, 164b, 164c, and 164d may be overlapped or nested in series around the periphery of the vascular seal 160″. In some variations, the amount of overlap between leaflets may generally correspond to the amount of radial contraction or radial expansion of the vascular seal 160″. For example, when the vascular seal 160″ is in the open state, the leaflets may be less overlapped than when the vascular seal 160″ is in the closed state.

In some variations, the vascular seal may maintain a peripheral seal against the inner wall of the cardiovascular lumen during both the closed and open states of the vascular seal, and a membrane of the vascular seal may include one or more additional sub-valves that prevent or permit the passage of fluid through the vascular seal in the closed or open states, respectively. For example, FIG. 6C is a schematic illustration of an example vascular seal 160′″ including a membrane 164 (e.g., similar to that described above with respect to FIG. 6A) including one or more flap valves 168. A flap valve 168 may, for example, include at least one aperture and at least one flap. A particular flap valve 168 may have one aperture and one flap, multiple apertures and one flap, or one aperture and multiple flaps. Pressure differential between upstream and downstream sides of the vascular seal 160′″ may, for example, cause movement of the flap(s) relative to the aperture(s) for each flap valve 168. In some variations, flap valves for the vascular seal may be similar to those described in greater detail in International Patent Application No. PCT/EP2023/059293, which is incorporated above by reference.

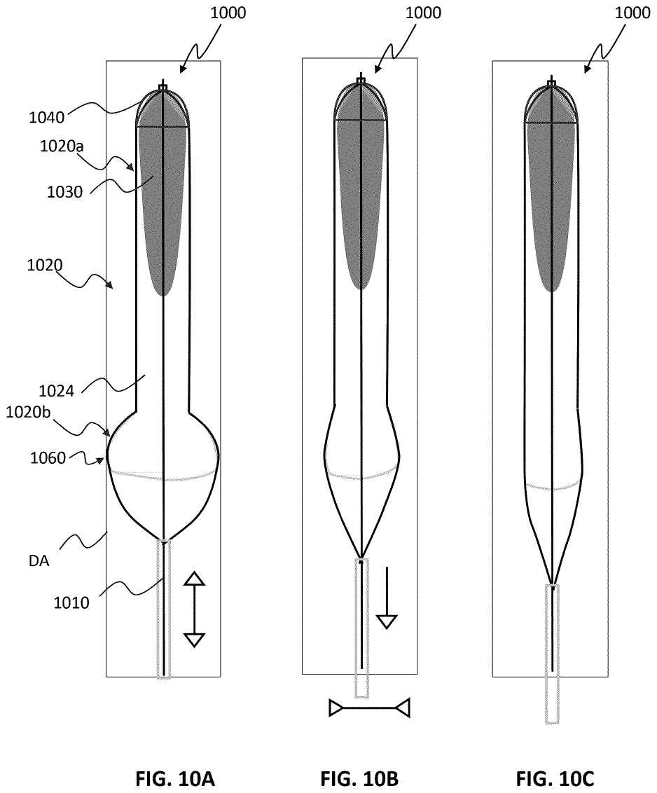

In some variations, a vascular seal may be integrally formed with a pump body of a pump arrangement. For example, FIGS. 10A-10C illustrate operation of an example circulatory assist device 1000 including a vascular seal 1060 extending from a proximal end of a pump body in the pump arrangement 1020. Similar to the circulatory assist device 100 and other variations described herein, the circulatory assist device 1000 may include a pump body with an inlet valve 1040, a volume displacement member 1030 in the pump body, and a catheter 110 including at least one lumen for conveying inflation fluid for expanding and contracting the volume displacement member 1030. However, the pump body in the circulatory assist device 1000 may include an outflow region having a bulbous or flared shape with an adjustable outer diameter to function as an integrated vascular seal 1060. The vascular seal 1060 may, for example, include a portion or an extension of a support structure of the pump body (e.g., nitinol or other shape memory material), and at least a portion of a membrane 1024 in the pump body may extend at least partially over the outflow region. The support structure and membrane of the outflow region may be configured to expand to an expanded diameter larger than the diameter of the remaining portion of the pump body so as to form a seal with the vessel wall. In some variations, the vascular seal 1060 may include a shape memory material configured to cause the vascular seal 1060 to self-expand to match the inner wall of the cardiovascular lumen (e.g., descending aorta (DA)), thereby forming a peripheral seal against the inner wall of the cardiovascular lumen. In some variations, the outflow region of the pump body may be configured to expand to a larger diameter under the pressure of blood flowing through the outflow region when the pump is operating. For example, the outflow region may be formed of an elastically distensible polymeric material, foldable fabric or tissue, or a series of flexible fabric segments connected to spokes or tines similar to an umbrella, in each case being expanded to a larger diameter to seal with the vessel wall when the pump is operating. FIG. 10A shows the circulatory assist device 1000 with the vascular seal 1060 in a closed state, forming the peripheral seal between the pump arrangement 1020 and the inner wall of the cardiovascular lumen.

The closed and open states of the vascular seal may be actively controlled. For example, closed and open states of the vascular seal may be actively controlled by controlling the longitudinal position of a proximal end of the vascular seal 1060. The longitudinal position of a proximal end of the vascular seal 1060 and/or outer diameter of the vascular seal 1060 may be controlled by one or more pull wires (not shown) coupled to the vascular seal 1060 and/or tethers (not shown) such as similar to that described above (e.g., with respect to FIG. 1B). For example, as shown in FIG. 10A, the longitudinal position of the proximal end of the vascular seal 1060 may be adjusted proximally and distally with one or more tethers in the catheter 1060. Proximal movement of the tethers (e.g., pulling) may cause elongation and/or radial contraction of the vascular seal 1060 (FIG. 10B), thereby transitioning the vascular seal 1060 to an open state (FIG. 10C) that permits fluid flow through the cardiovascular lumen around the pump arrangement 1020. Additionally or alternatively, distal movement (e.g., pushing) of the tethers may cause radial expansion of the vascular seal 1060, thereby transitioning the vascular seal 1060 to a closed state (FIG. 10A) that forms the peripheral seal between the pump arrangement 1020 and the cardiovascular lumen. Other aspects of the example circulatory assist device 1000 are described in further detail below.

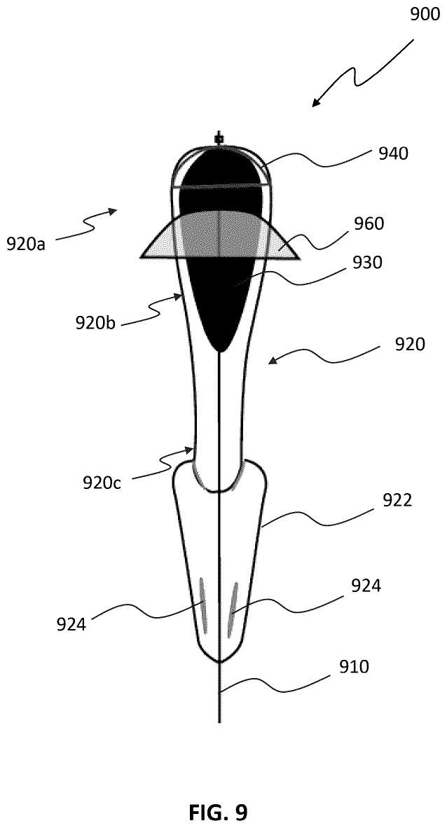

The vascular seal may be located at any suitable axial location along the circulatory assist device. For example, in some variations the vascular seal may be arranged proximal to the volume displacement member or other pump (e.g., FIGS. 1C, 5A, 7A, 8, 10A-10C), such as adjacent the outflow region of the pump arrangement or distal to the outflow region of the pump arrangement (e.g., proximal end of an outflow nozzle region of the pump arrangement). As another example, in some variations the vascular seal may be arranged adjacent to the volume displacement member or other pump (e.g., FIG. 9), such as adjacent to a central or proximal region of the volume displacement member or other pump. In some variations, the vascular seal may be positioned at an axial location between an inflow region of the pump arrangement and the outflow region of the pump arrangement.

It should be understood that in other variations, various features of these example vascular seals and/or other aspects of the example circulatory assist devices may be combined in any suitable manner. For example, in some variations, a vascular seal may include multiple leaflets (as described above with respect to FIG. 6B) and reinforcement members (as described above with respect to FIG. 6A). Furthermore, any of the example vascular seals may be arranged at any suitable longitudinal location along the circulatory assist device (e.g., proximal to the pump, adjacent to the pump, etc.).

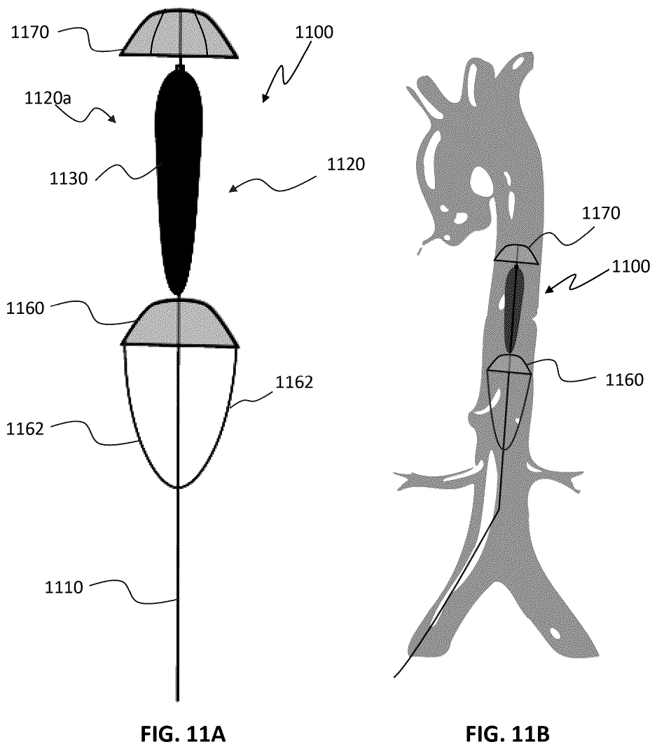

Although many variations of the circulatory assist device described herein include a pump arrangement having a pump region with a conduit, in some variations the pump region may omit a conduit. For example, in some variations the pump region may include or be defined at least in part by one or more vascular seals each configured to form a respective peripheral seal against an inner wall of a cardiovascular lumen. Each vascular seal can be similar to any of the vascular seals described herein. FIG. 11A illustrates an example variation of a circulatory assist device 1100 including a pump arrangement 1120, a first vascular seal 1160 at a proximal portion of the pump region, and a second vascular seal 1170 at a distal portion of the pump region. As shown in FIG. 11B, when the circulatory assist device 1100 is placed in a cardiovascular lumen such as in a descending aorta, the first vascular seal 1160 may be configured to form a first peripheral seal against the lumen wall, and the second vascular seal 1170 may be configured to form a second peripheral seal against the lumen wall. A volume displacement member 1130 may be arranged between the first and second vascular seals, such that when the circulatory assist device 1100 is placed in a cardiovascular lumen, the volume displacement member 1130 is positioned between the first and second peripheral seals. Accordingly, the volume displacement member 1130 may be positioned in a functional pump region between the first and second vascular seals. For example, the cardiovascular lumen wall may at least in part define a volume for pumping fluid.

When the circulatory assist device 1100 is deployed as shown in FIG. 11B, the first and second vascular seals may be configured to permit fluid flow from a distal end of the device 1100 to a proximal end of the device 1100, while substantially preventing fluid flow in the reverse direction (from a proximal end of the device 1100 to a distal end of the device 1100). Furthermore, one or both of the first vascular seal 1160 and the second vascular seal 1170 may include support feature(s) to help prevent inversion, such as tethers 1162 (e.g., similar to tethers 162 described above) and/or reinforcement members in the vascular seal. The circulatory assist device 1100 is shown in FIG. 11B deployed with its full length between the first and second vascular seals located in an unbranching portion of a cardiovascular lumen (e.g., a region without vessel take-offs). However, it should be understood that in some variations the circulatory assist device 1100 may be deployed in any suitable cardiovascular lumen. For example, in some variations the circulatory assist device 110 may be deployed in a descending aorta, with the first vascular seal 1160 located inferior to a renal or hepatic takeoff, while the second vascular seal 1170 located superior to a renal or hepatic takeoff. In this example blood flow through the renal or hepatic takeoff(s) may be assisted (e.g., enhanced) by the circulatory assist device 1100, in addition to or alternative to assisting blood flow through the descending aorta with the circulatory assist device 1100.

In some variations, a circulatory assist device may be similar to circulatory assist device 1100, except it may have only a single vascular seal. For example, a circulatory assist device may be similar to circulatory assist device 1100 shown in FIG. 11A, except that it may omit the second vascular seal 1170. A single vascular seal 1160 located at a distal portion of the pump region may, for example, be suitable in instances when the volume displacement member 1130 creates sufficient fluid momentum. In some variations, conveyance of fluid through the outflow region may be sustained at least partially due to created and maintained momentum of a fluid column along a flow axis of the conduit that extends between an inflow region and the outflow region of the pump arrangement.

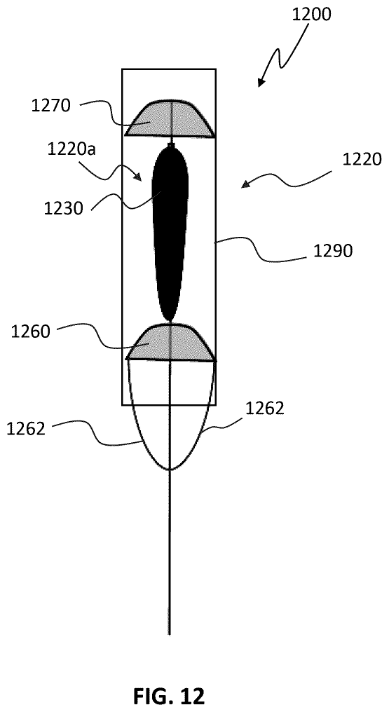

Although the circulatory assist device 1100 may be operable in a tubular cardiovascular lumen (e.g., descending aorta) where the lumen wall at least in part defines a volume for pumping in the pump region, the circulatory assist device 1100 may be modified to be suitable for placement in other locations, such as a left ventricle. For example, FIG. 12 illustrates an example circulatory assist device 1200 that is similar to the circulatory assist device 1100, except the circulatory assist device 1200 may further include a hollow tube 1290 configured to surround at least a portion (e.g., pump region) of the pump arrangement 1220. For example, the hollow tube 1290 may be configured to surround at least a portion of the pump arrangement 1220 including the first vascular seal 1260 and the second vascular seal 1270. In some variations, the hollow tube 1290 may include a flexible material, such as that similar to the membrane 124 described above with respect to the pump body. The hollow tube 1290 may function as a pump body or conduit for the circulatory assist device 1100, when the circulatory assist device 1100 is placed in non-tubular treatment locations, such as the left ventricle.

In some variations, a circulatory assist device may omit a vascular seal. For example, in some variations the circulatory assist device may include a pump body configured to diverge or widen the outflow of fluid from the circulatory assist device, without a vascular seal. As described elsewhere herein, shaping the outflow of fluid to have a wider profile may help reduce outflow fluid velocity, thereby reducing or preventing regurgitation of blood and reducing complications such as hemolysis.

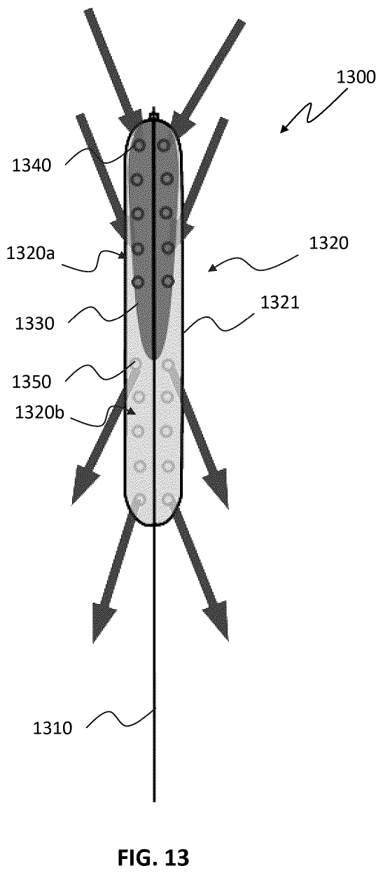

For example, FIG. 13 illustrates an example circulatory assist device 1300 having a pump arrangement 1320 with an elongated pump body 1321. The pump body 1321 may include a support and one or more membranes adjacent to the support, similar to the pump arrangement 120 described above with respect to FIG. 3. However, as shown in FIG. 13, the pump body 1321 further includes a plurality of inlets 1340 and a plurality of outlets 1350. The plurality of outlets 1350 may be arranged axially along and/or circumferentially around a proximal outflow region, so as to create wide pressure waves in the outflow of fluid from the pump body 1321. These wide pressure waves may generate wall-like fluid flow out of the pump body 1321 over a larger surface area, rather than a narrower, more focused outflow jet of fluid. Although the circulatory assist device 1300 omits a vascular seal, in some variations the circulatory assist device 1300 may still include a vascular seal (e.g., similar to those described herein) configured to form a peripheral seal between the pump arrangement 1320 and an inner wall of a cardiovascular lumen.

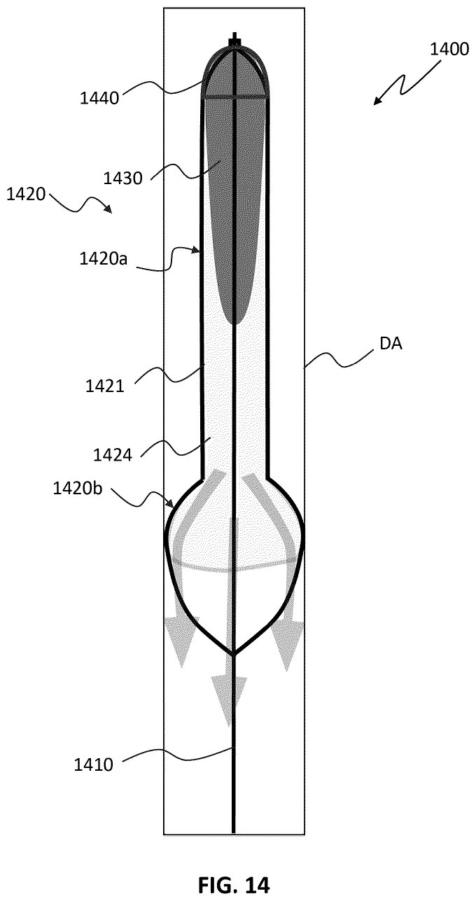

As another example, FIG. 14 illustrates an example circulatory assist device 1400 with a pump body configured to diverge or widen the outflow of fluid from the circulatory assist device 1400. The circulatory assist device 1400 may include a pump arrangement 1420 with a pump body 1421 having a wider or bulbous outflow region 1420b. The wider outflow region 1420b may be configured to expand (e.g., due to being self-expandable or expanded with a device such as a balloon) to a diameter approximating the diameter of the cardiovascular lumen in which the device 1400 is placed. When expanded in this manner, the outflow region 1420b may be configured to diverge the outflow of fluid to a diameter similar to the diameter of the cardiovascular lumen in which the device 1400 is placed, thereby slowing exiting fluid velocity and reducing the likelihood of fluid regurgitation and hemolysis, etc.

### D. Catheter

The catheter 110 may have specific features allowing or enhancing the high frequency operation of the circulatory assist device 100, and/or to optimize inert flow for the volume displacement member 130 (e.g., in variations in which the volume displacement member 130 is a balloon).

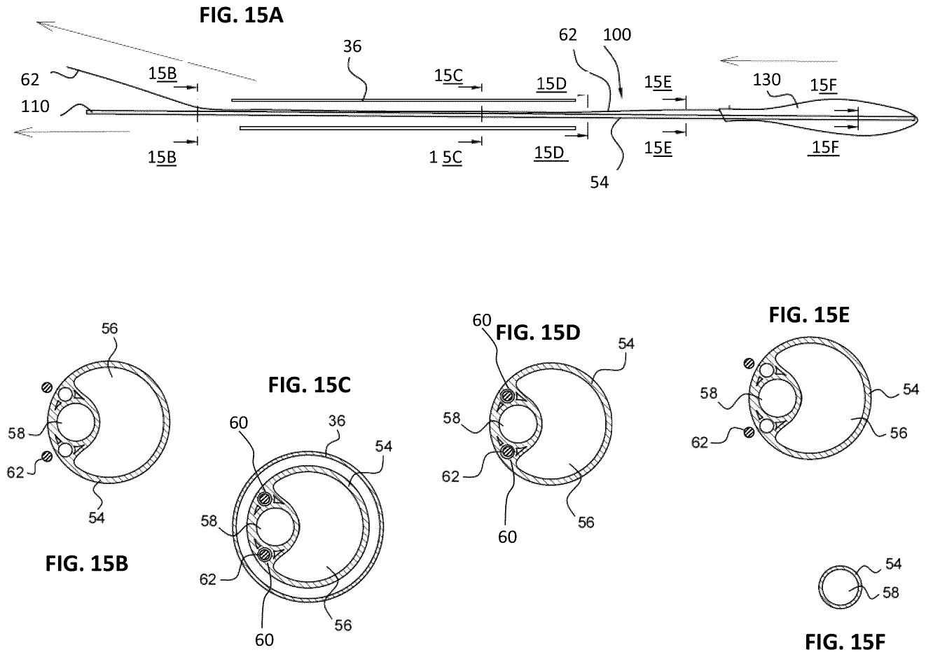

FIGS. 15A-15F depict an example variation of a catheter 110 that is suitable for use with a volume displacement member 130 including a balloon. Variations of the catheter 110, such as for use with other kinds of pumps 130′, may additionally or alternatively include other features such as lumen(s) for control wires carrying power and/or control signals to an impeller pump. In some variations, as shown in FIG. 15A which shows the catheter 110 extending through a delivery sheath 36, the catheter 110 may include a balloon shaft 54 having an inflation lumen 56. The inflation lumen 56 may, for example, have a cross-sectional flow area of between 1 mm² and 20 mm². This corresponds to an inner diameter of generally between about 0.5 mm and about 5 mm, which allows a proper balance between flow resistance for the inert fluid and further characteristics of the catheter parts, such as bending radius, kinking resistance, etc. In some variations, the inflation lumen 56 is not subdivided, since the resistance will go up when the cross-sectional area is distributed over different channels. The shape of the inflation lumen 56 may be configured to have the lowest resistance possible, while leaving room in the catheter for a potential guidewire, pull wires for device retrieval, and/or any potential sensors.

In some variations, as shown in FIGS. 15B-15E, the balloon shaft 54 includes three or more lumens, including an inflation lumen 56 (which may be largest in diameter among the lumens), a guidewire lumen 58, and one or multiple wire lumens 60 for pull wire(s) 62 for retrieval of the pump body 121 and/or controlling one or more vascular seals. The catheter 110 may include one or more sensors at or near its distal end. For example, the catheter 100 may include one or more pressure transducers for measuring pressures within the conduit of the pump body 121, or in the heart outside the pump body 121, electrical sensors such as heart rate sensors, and/or or other sensors. The balloon shaft 54 may include lumens for wires to any such sensors.

In some variations, a distal section of the catheter 110 may have a wider diameter than a proximal section of the catheter, the proximal section of the catheter being farther from the balloon-based volume displacement member 130 than the distal section of the catheter. This may allow the inflation lumen to be larger in the distal section of the catheter 110 than in the proximal section of the catheter 110, and thereby may lower friction of the inert inflation fluid passing through the larger distal section of the catheter 110. The distal section may also be configured to be positioned in the larger vessels closer to the heart relative to the point of percutaneous introduction, such as in the aorta. As an illustrative example, a distal section of the catheter 110 may be 60 mm long with a diameter of about 2.5 mm, and a proximal section of the catheter (in the aorta, femoral area, and outside the patient) may be about 1200 mm long and a diameter of about 2.2 mm or less. In some variations, the overall insertable length of the catheter 110 may be configured to extend from a femoral puncture site (e.g., from an insertion site at the femoral artery) of the patient, through the aorta, and into the left ventricle.

In some variations, the catheter 110 may include a plurality of catheter sections with different diameters. The wider diameter sections may, for example, be configured for placement in areas where blood flow is not obstructed (e.g., peripheral arteries) or where they remain outside of a patient during operation. In an illustrative example, a first section (within the pump body 121) is about 60 mm long with a diameter of 2.2 mm, a second section (in the aorta area) is 800 mm long with a diameter of 3 mm, a third section (in the femoral area) is 400 mm long and a diameter of 2.5 mm, and a fourth section (outside the patient) has a length of 750 mm and a diameter of 4 mm.

In some variations the catheter 110 may include a stiff material selected to provide a low flow resistance (i.e., a low impedance to the inflation and deflation pressures during operation) as well as kink resistance. For example, in some variations the catheter may include a nylon material with a wall thickness of between 0.1 mm and 0.3 mm (e.g., 0.2 mm). Dedicated catheter material and dimension choices allow preservation of the radial shape, while being sufficiently flexible in the longitudinal direction. The shaft may have a high radial stiffness, achieved by high durometer material such as nylon 12 Pebax or polyimide of 72D or higher wall thickness, which may be reinforced with a braid or coil (e.g., wire or ribbon). The high durometer may, for example, help to facilitate rapid transport of helium. The durometer may vary over the shaft length to accommodate the curvature of the vasculature or ascending aorta.

Furthermore, in some variations the catheter 110 may include a thermally insulative coating layer over its exterior. This may help to maintain the (relative) low temperature of the inert fluid, such as helium, thereby giving it higher density and allowing higher flow velocities. Additionally or alternatively, the control system 2 (further described below) may be provided with an active cooling subsystem for controlling the temperature of the inert fluid delivered to the catheter 110 during operation. In some variations, the inflation fluid may be cooled and maintained at a temperature between −20 degrees Celsius and 20 degrees Celsius.

The catheter 110 and/or the pump body 121 may be configured to minimize vibration or oscillation when operated at high frequency. When fluid exits the pump body 121, the resulting thrust may lead to a force in the opposite direction, which is a counterforce that can potentially move the pump body 121 from its equilibrium position to a position deeper into the left ventricle. Once the pump stroke has been completed, the pump body 121 may seek to return into its equilibrium position, driven by the pull from the catheter 110 and the push from the distal tip of the circulatory assistance device 100. The size, geometry, and stiffness of balloon shaft 54 may be selected such that it acts to dampen this motion of the pump body 121. Further, by operating the volume displacement member 130 (e.g., balloon) at a sufficiently high frequency, the next pump stroke will happen before the pump body 121 has time to relax and return to its equilibrium position. In this case, the device will be “trapped” in a position away from its equilibrium position. The higher the frequency, the less time the pump body 121 has to move back towards its equilibrium position, and the more stable the device tip will be.

### E. Control System

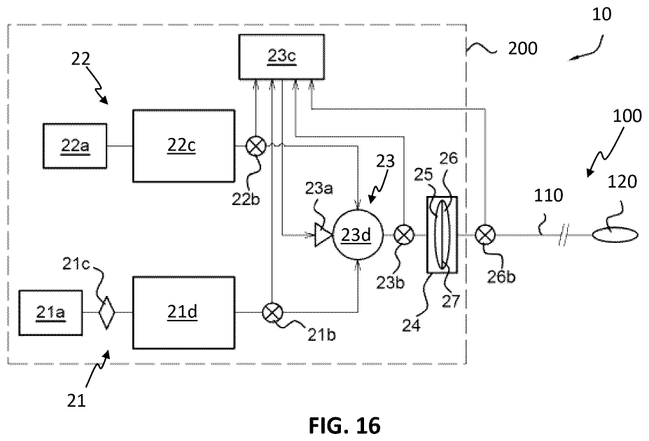

FIG. 16 shows a schematic diagram of details of an example control unit 2 of a circulatory assist system 10 according to the present technology. The control unit 2 may be configured to operate a balloon-style volume displacement member that is operable in an inflation phase and a deflation phase. For example, the control unit 2 may be configured to deliver an inflation fluid and regulate pumping parameters to provide the desired high blood flow rates from a very compact pump. The control unit may be configured to deliver any one or more selected inflation fluids at pressures and temperatures selected to allow cyclical expansion of the volume displacement member at a high frequency. For example, the control unit 2 may cyclically expand the volume displacement member 130 at a frequency of at least about 300 beats per minute, at least about 1000 beats per minute, at least about 1500 beats per minute, at least about 2000 beats per minute, at least about 2500 beats per minute, at least about 3000 beats per minute, at least about 5000 beats per minute, or between about 1000 beats per minute and about 10,000 beats per minute (e.g., between about 1500 and about 3,000 beats per minute). In some variations, the inert fluid may be a low viscosity fluid such as helium or carbon dioxide gas, to minimize friction with inside walls of the catheter assembly. Helium has the additional advantage of having a low density, therefore a lower mass inertia, allowing higher inflation frequencies.

In some variations, the control unit may allow user adjustment or tuning of the frequency of cyclical expansion of the volume displacement member so that an appropriate frequency can be selected by the user for a particular patient and procedure, or the frequency can be changed during a procedure according to the patient's needs. Additionally, the control unit 2 may allow for adjustment of the volume displaced by the volume displacement member (e.g., its volume in the fully contracted low-volume state, in the fully expanded high-volume state, or both).

In some variations, the volume change of the volume displacement member may be generated by changing the pressure of the volume enclosed by volume displacement member, such as an inflatable balloon. This may be achieved by pressurizing and depressurizing the enclosed volume through the connecting inner lumen of the catheter shaft. Accordingly, in some variations, the control unit 2 may include a high-pressure pump arrangement 21, a low-pressure pump arrangement 22, and a switching arrangement 23 connected to the high-pressure source 21. The low-pressure pump arrangement 22, the catheter 110, and the switching arrangement 23 may be arranged to alternately connect the high-pressure source 21 and the low-pressure source 22 to the catheter assembly 3. This may allow the control unit 2 to use suitable hydraulic/pneumatic control components, with reliable and robust (bedside) operation. As shown in FIG. 16, the high-pressure source 21 may be implemented as a combination of a high-pressure buffer 21d, high pressure compressor 21a and regulator 21c (and optionally high-pressure sensor 21b). The low-pressure source 22 may be implemented as a combination of a vacuum buffer 22c, vacuum pump 22a and low-pressure sensor 22b. The switching arrangement 23 may be implemented as a combination of a 3-way valve unit 23 d controlled via switch valve 23 a using, e.g., a controller 23 c. The controller 23c receives signals from the high-pressure sensor 21b, low pressure sensor 22b, and switch pressure sensor 23b, in order to properly drive the switch valve 23a. Connection to the catheter assembly 3 is implemented via a safety driver 24. To allow high frequency operation of the 3-way valve unit 23 d, a valve design may be selected to have an optimal flow through the valve, with minimal turbulence, high switch speed and low leak rate.

In some variations, the control unit 2 may further include a safety driver 24 having a source side chamber 25 and a catheter side chamber 26 separated by a safety diaphragm 27. This may allow the use of a pneumatic/hydraulic part of the control unit 2, separated from an inert gas side part of the control unit 2, to be connected to the catheter assembly 3, minimizing the volume of inert gas needed in the heart assist system 100. The safety driver 24 accommodates the actuation of the inert gas circuit, by compressing and expanding the inert gas circuit at the catheter side chamber 26 of the safety driver 24. The actuation speed of the safety driver is sufficient to e.g., provide a pressure difference between +800 to −760 mmHg, in a volume of, e.g., 20 ml (typical range is 5-70 ml) within 5-200 ms.

In some variations, the control unit 2 may be arranged to connect the high-pressure pump arrangement 21 to the catheter 110 during an inflation phase and to connect to the low-pressure pump arrangement 22 during a deflation phase. This duty cycle may be altered with variable counterpressure. With higher counterpressure, there may be a need for more inflation time, while the deflation may be faster, when the environmental pressure also reduces the balloon volume.

The control unit 2 furthermore may be arranged to respond to sensor data or user input. For example, the control unit 2 may be responsive to certain sensed counter pressures, or simply to create an additional pulsatile flow, by altering the speed of the inflation. For example, the control unit 2 may respond to ECG triggers by switching between a lower frequency (e.g., 300 beats per minute) and a higher frequency (e.g., 5000 beats per minute), operating the circulatory assist device 100 only during diastole or only during systole, or briefly pausing inflation or deflation at a specific detected moment in the cardiac cycle.

In some variations, the safety driver 24 (or safety chamber) may be sized relative to the total volume of the inert gas (e.g., helium) circuit. The safety diaphragm 27 may be movable so as to alter the volume of the inert gas circuit. For example, by moving the safety diaphragm 27 into the source side chamber 25, the total volume of the inert gas circuit can be enlarged, thus depressurizing the inert gas circuit. Furthermore, by moving the safety diaphragm 27 in the opposite direction, the total volume of the inert gas circuit may be reduced, and thereby pressurized. With this actuation, pressures can be obtained in the inert gas circuit of, for example, between 600 mmHg and −600 mmHg in a 130 cm long catheter assembly 3 with a cross section area of, e.g., 3 mm² in order to inflate and deflate the volume displacement member within 10 ms.

To further optimize the translation of pressure from the safety diaphragm 27 to the volume displacement member 130, the length of catheter assembly 3 can be minimized. Therefore, the safety diaphragm 27, and other components of the control unit 2 may be adapted to be included in a bedside control unit, such as by using external versions of the high-pressure source 21 and low-pressure source 22. The bedside control unit can then be mounted to the bed at a distance of 20-100 cm away, in some cases less than 30 cm away, from the vascular access site on the patient.

In some variations, as shown in FIG. 16, the inert gas circuit between the safety diaphragm 27 and the volume displacement member 130 may be provided with an inert gas pressure sensor 26b, the signal of which can be provided to the controller 23c for user information, driver control and/or failure detection functions. For example, in the control unit, a continuous check for the pressure waveforms may be applied, such as to detect a gas leak or kinking/obstruction of the catheter 110. For example, a pressure drop below a predetermined value may indicate a possible inert gas leak, in which case the circulatory assist device 100 may be stopped or switched to a vacuum/low pressure control mode. As another example, a pressure drop above a predetermined valve may indicate possible kinking of the catheter or other obstruction of flow in the catheter. Suitable alerts or notifications may be communicated to a user (e.g., on a display, audio alerts, etc.) to prompt suitable remedial actions. More advanced software may be used in the control unit to determine the ventricular pressure from the inert gas (balloon) pressure.

Furthermore, in some variations, the safety driver 24 may be cooled/heated (e.g., to about 10 degrees Celsius) in order to preserve the material properties, safety, and/or inert gas flow speed. As an alternative to this indirect temperature control of the inert fluid in the catheter 110 during operation, the inert fluid temperature may be controlled using an active fluid cooling subsystem. In some variations, the inert gas circuit may be provided with an automatic filling system. For example, to ensure stable helium concentration, every two hours (or periodically with an interval between 30 min and 4 hours) the helium system may be emptied and replaced with an automatic injection of a new volume of helium.

### II. Methods of operation

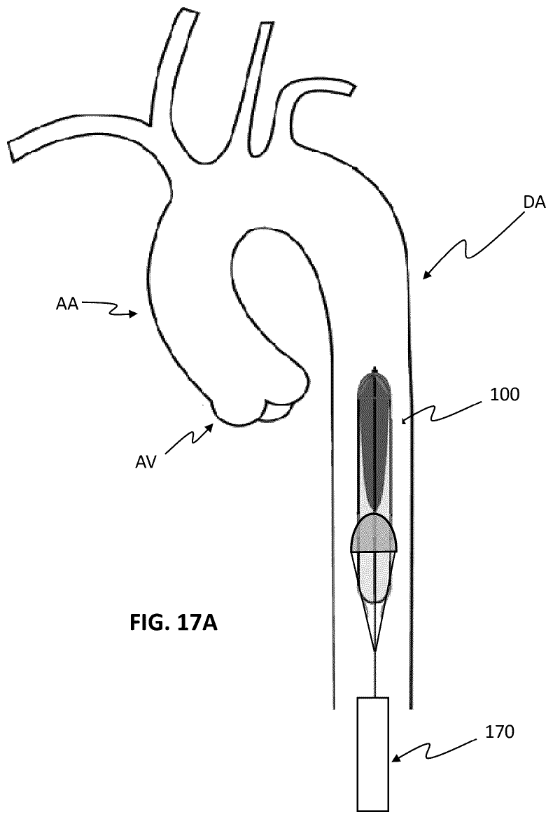

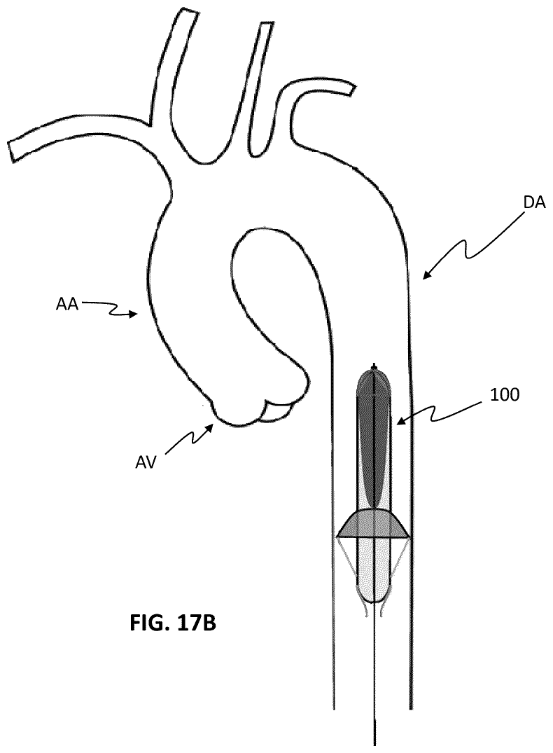

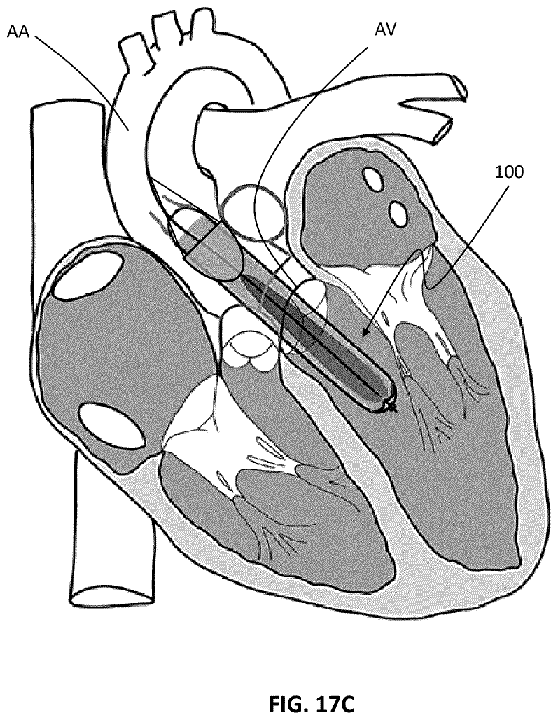

As described herein, any of the circulatory assist devices described herein in accordance with the present technology may be placed in a treatment location in one or more cardiovascular lumens in a patient, to provide circulatory assistance. An example method of using a circulatory assist device is described below with reference to FIGS. 17A-17C. Although FIGS. 17A-17C depict operation of a particular variation of the device similar to circulatory assist device 100 shown in FIG. 1B, it should be understood that the various processes in the method may be performed with respect to any other variation of circulatory assist device in accordance with the present technology.

In some variations, a method of operating a circulatory assist device may include positioning a circulatory assist device in a cardiovascular lumen of a patient, where the circulatory assist device includes a pump arrangement with a pump region configured to receive blood and an outflow region, and a pump, and a vascular seal arranged adjacent the pump arrangement. The method may further include forming a peripheral seal between the pump arrangement and an inner wall of the cardiovascular lumen with the vascular seal, and operating the pump to drive blood through the outflow region and into the cardiovascular lumen.

FIG. 17A partially illustrates an example process for positioning a circulatory assist device in a descending aorta of a patient. Generally, a circulatory assist device 100 may be percutaneously introduced into the cardiovascular system of a patient through a peripheral insertion site, such as a femoral artery. The circulatory assist device 100 may, for example, be in a radially constricted delivery configuration, and may be radially constrained in a delivery sheath 170. The sheath 170 and the circulatory assist device 100 may be advanced into the descending aorta to a treatment location (such as with a guidewire (not shown)), whereupon the delivery sheath 170 may be proximally retracted to expose the circulatory assist device 100. In some variations, the positioning of the sheath 170 and the circulatory assist device 100 may be performed without imaging guidance.

Once exposed, the circulatory assist device 100 may become radially expanded. For example, the circulatory assist device 100 may self-expand or may be expanded with a separate balloon device or other suitable expandable device. The vascular seal (if present on the circulatory assist device 100) may also expand to its closed state as shown in FIG. 17B, thereby forming a peripheral seal between the pump arrangement and inner wall of the descending aorta. FIG. 17B depicts the circulatory assist device 100 in its active state, in which the circulatory assist device 100 receives blood through an inlet and accelerates flow via cyclical operation of the volume displacement member arranged in the circulatory assist device 100. Further operation of the circulatory assist device 100 can involve transitioning between an active state and a passive state as shown and described above with reference to FIGS. 2A and 2B. In some variations, cyclical operation of the volume displacement member to pump blood with the circulatory assist device 100 in the descending aorta may provide a circulatory assistance treatment at a first intensity level. In some variations, treatment can continue with the circulatory assist device 100 positioned in the descending aorta, and may be removed from the patient by radially collapsing the circulatory assist device 100 (e.g., with pull wires) and/or withdrawing the device 100 back into a sheath 170, then proximally withdrawing the device 100 and sheath 170 from the patient.

However, in some variations, treatment intensity may be escalated by repositioning the circulatory assist device 100 to a second location and operating the pump while the circulatory assist device 100 is at the second location. For example, in some variations the method may further include repositioning the circulatory assist device 100 at least partially in a left ventricle of the patient to increase circulatory support of the patient. For example, the circulatory assist device 100 may be positioned in a transvalvular location such as that shown in FIG. 17C, where a distal portion of the circulatory assist device 100 is in the left ventricle and a proximal portion of the circulatory assist device 100 is in the aorta (e.g., ascending aorta (AA)), and the circulatory assist device 100 crosses a plane of the aortic valve (AV). In this location, the vascular seal (if present) of the circulatory assist device 100 may be radially collapsed into a passive state. Circulatory assistance at this transvalvular location may provide a circulatory assistance treatment at a second intensity level that is higher than the first intensity level. When treatment is no longer desired at this higher intensity level, treatment may subsequently be decreased (e.g., for weaning purposes) by repositioning the circulatory assist device 100 in the descending aorta for providing treatment at a lower level (e.g., at or around the first intensity level). When treatment is no longer desired, the circulatory assist device 100 may thereafter be withdrawn proximally and removed from the patient.

In some variations, initial treatment in the descending aorta (FIGS. 17A and 17B) may be skipped, and the circulatory assist device 100 may be directly placed at least partially in the left ventricle (e.g., at a transvalvular location). Treatment may subsequently be decreased (e.g., for weaning purposes) by repositioning the circulatory assist device 100 in the descending aorta. When treatment is no longer desired, the circulatory assist device 100 may thereafter be withdrawn proximally and removed from the patient.

Similarly, in some variations, a circulatory assist device may be positioned and operated in a pulmonary artery instead of a descending aorta, to provide cardiovascular support to the right ventricle, for example. The circulatory assist device may additionally or alternatively be positioned (or repositioned) and operated in the right ventricle for providing a higher level of cardiovascular support compared that provided when the device is placed in the pulmonary artery.

### III. Examples

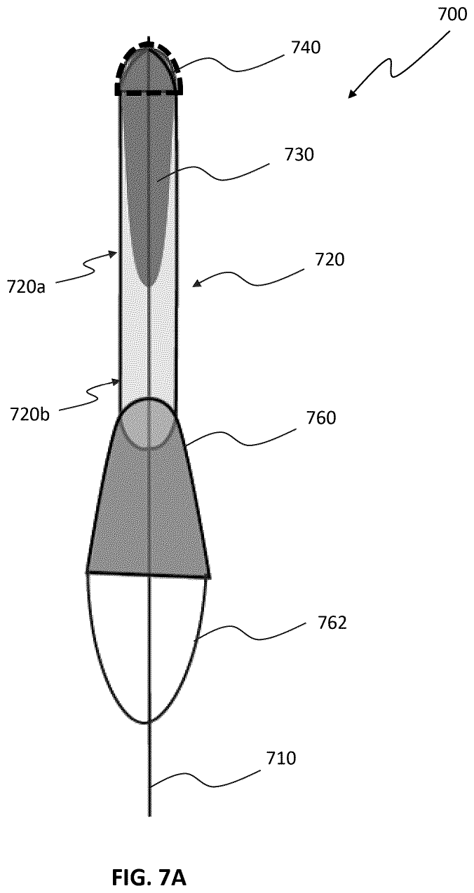

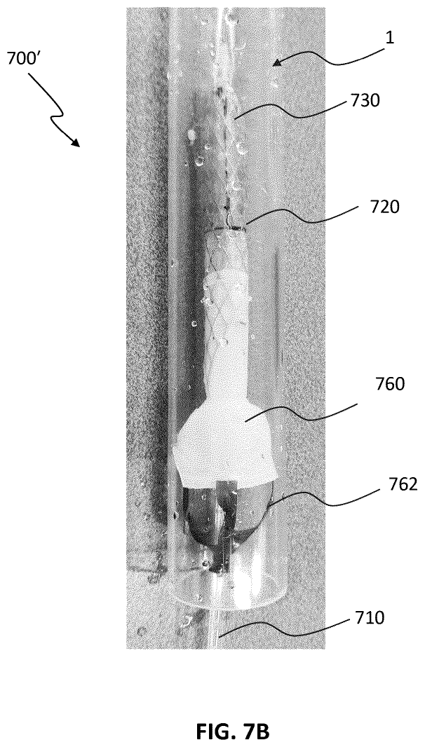

FIGS. 7A-14 illustrate various examples of circulatory assist devices, especially various configurations of vascular seals. Although these examples are shown and primarily described as having a pump including a balloon-type volume displacement member, it should be understood that in other examples, a circulatory assist device may be similar to any of those shown and described with respect to FIGS. 7A-14, except with a different type of pump (e.g., impeller pump). For example, any of the examples of FIGS. 7A-14 may include an impeller pump or other suitable kind of pump, in combination with the pictured and described vascular seal of those examples (e.g., vascular seal 760, 860, 960, 1060, 1160, 1260 and/or 1270) and/or the pump body of those examples.

FIG. 7A illustrates an example circulatory assist device 700 including a pump arrangement 720 with an elongated pump body having a conduit. The conduit may include a distal inlet valve 740 configured to convey fluid (e.g., blood) into a pump region 720a. A volume displacement member 730 may be arranged in the pump region 720a, and may be configured to drive received fluid toward and through an outflow region 720b of the pump arrangement 720. The volume displacement member 730 may include a balloon that may be cyclically inflated and deflated with an inflation fluid circulated via one or more lumens in a catheter 710. A vascular seal 760 may be integrally formed with or attached to the outflow region 720b of the pump arrangement 720. As shown in FIG. 7A, the vascular seal 760 may have a generally outwardly flared shape (e.g., skirt shape) configured to help diverge outflow of fluid to a wider outflow profile, thereby functioning as an outflow nozzle in some instances. One or more tethers 762 (e.g., pull wires or flexible strips) may be attached to the vascular seal 760 (e.g., proximal end or peripheral edge of the vascular seal 760) and pulled proximally to radially collapse and/or elongate the vascular seal 760. The tethers 762 may additionally or alternatively help prevent inversion of the vascular seal 760.

FIG. 7B illustrates an example circulatory assist device 700′ constructed in a manner similar to circulatory assist device 700 described above with respect to FIG. 7A. The circulatory assist device 700′ is shown in FIG. 7B in an active state with the vascular seal 760 radially expanded against an inner surface of a polymer tube 1, which serves as a proxy for a descending aorta or other tube-like cardiovascular lumen. Four tethers 762 (e.g., flexible polymer strips) each has a first end attached to a peripheral proximal edge of the vascular seal 760 with an approximately equal circumferential distribution (e.g., 90 degrees apart), and a second end connected to pull wires or a member that can pass through a catheter 710. As described above, the tethers 762 may be configured such that proximal movement of the tethers 762 causes radial collapse and/or elongation of the vascular seal 760, such as for deactivation of the vascular seal 760 (e.g., for repositioning or withdrawal of the circulatory assist device 700′).

FIG. 8 illustrates an example circulatory assist device 800 including a pump arrangement 820 with an elongated pump body having a conduit. The conduit may include a distal inlet valve 840 configured to convey fluid (e.g., blood) into a pump region 820a. A volume displacement member 830 may be arranged in the pump region 820a, and may be configured to drive received fluid towards and through an outflow region 820b of the pump arrangement 820. The volume displacement member 830 may include a balloon that may be cyclically inflated and deflated with an inflation fluid circulated via one or more lumens in a catheter 810. A vascular seal 860 may be integrally formed with or attached to the pump body at a location proximal to a proximal end of the volume displacement member 830, though in some examples may be located in any suitable location. The vascular seal 860 may include a membrane that is supported with one or more reinforcement members and/or includes one or more tethers (e.g., similar to tethers 762 described with respect to FIG. 7A) to help prevent inversion of the vascular seal 860. The tethers (not shown) may additionally or alternatively be used to radially collapse and/or elongate the vascular seal 860.

The pump arrangement 820 may further include an outlet valve 850 configured to convey fluid into an outflow structure 822 that includes a flexible chamber for buffering fluid exiting the pump arrangement 820. The outflow structure 822 may include multiple (e.g., at least two, or at least four) outlets 824 configured to convey fluid in a radial direction, such as orthogonal to a longitudinal axis of the outflow structure 822. Such radially-oriented outlets 824 may be generally equally radially distributed around the outflow structure 822 (e.g., four outlets arranged about 90 degrees apart) to help diverge flow to a wider outflow profile.

FIG. 9 illustrates an example circulatory assist device 900 that is similar to the circulatory assist device 800 described above with respect to FIG. 8, except as described below. For example, the circulatory assist device 900 includes a pump arrangement 920 with an elongated pump body having a conduit. The conduit may include a distal inlet valve 940 configured to convey fluid (e.g., blood) into a pump region 920a. A volume displacement member 930 may be arranged in the pump region 920a, and configured to drive received fluid towards and through an outflow region 920b of the pump arrangement 920. The volume displacement member 930 may include a balloon that may be cyclically inflated and deflated with an inflation fluid circulated via one or more lumens in a catheter 910. The circulatory assist device 900 may include an outflow structure 920 that can be similar to the outflow structure 820 described above.

However, in the circulatory assist device 900, a vascular seal 960 may be integrally formed with or attached to the pump body at a location adjacent to the volume displacement member 930 (e.g., between distal and proximal ends of the volume displacement member 930). Additionally, the diameter of the conduit of the pump arrangement 920 may vary along the length of the conduit. For example, as shown in FIG. 9, the pump region 920a may have a larger diameter than the rest of the conduit, and may accommodate a generally teardrop-shaped volume displacement member 930. Furthermore, the circulatory assist device 900 may omit an outlet valve configured to convey fluid into the outflow structure 920.

FIGS. 10A-10C illustrate an example circulatory assist device 1000 in various states. As shown in FIG. 10A, the circulatory assist device 1000 may include a pump arrangement 1020 with an elongated pump body having a conduit. The conduit may include a distal inlet valve 1040 configured to convey fluid (e.g., blood) into a pump region 1020a. A volume displacement member 1030 may be arranged in the pump region 1020a, and may be configured to drive received fluid towards and through an outflow region 1020b of the pump arrangement 1020. The volume displacement member 1030 may include a balloon that may be cyclically inflated and deflated with an inflation fluid circulated via one or more lumens in a catheter 1010.

When the circulatory assist device 1000 is in an active state, the outflow region 1020b may have a maximum expanded diameter that is larger than the pump region 1020a. For example, the outflow region 1020b may have a bulbous shape. A membrane 1024 of the pump body may extend at least partially along the outflow region 1020b (e.g., extending at least to where the outflow region 1020b has its maximum diameter), such that the membrane 1024 and the enlarged shape of the outflow region 1020b may help diverge outflow of fluid to a wider outflow profile. Accordingly, the outflow region 1020b may function as an outflow nozzle in some instances. Additionally or alternatively, the outflow region 1020b may function as a vascular seal 1060, in that in the active expanded state, the outflow region 1020b may be configured to form a peripheral seal against a surrounding lumen wall (e.g., of a descending aorta (DA)). The vascular seal 1060 may be deactivated via proximal actuation of one or more tethers (e.g., wire, tube, etc.) coupled to a proximal portion of the vascular seal 1060, where such proximal actuation may cause radial contraction and/or elongation of the outflow region 1020b (FIGS. 10B and 10C). Additionally or alternatively, the vascular seal 1060 may be activated via distal actuation of one or more such tethers. Such distal actuation of the tethers may be manual and/or supported by a passive biasing mechanism (e.g., spring). In some other examples, the vascular seal 1060 may additionally or alternatively be activated via self-expansion (e.g., the outflow region 1020b may include shape memory material).

FIG. 11A illustrates an example circulatory assist device 1100 including a pump arrangement 1120 with a pump region 1120a configured to receive fluid (e.g., blood). A volume displacement member 1130 may be arranged in the pump region 1120a and configured to drive received fluid towards and through an outflow region of the pump arrangement 920. The volume displacement member 1130 may include a balloon that may be cyclically inflated and deflated with an inflation fluid circulated via one or more lumens in a catheter 1110. A distal vascular seal 1170 may be arranged distal to the volume displacement member 1130 and may function in some instances as an inlet valve for conveying fluid to the pump arrangement 1120. A proximal vascular seal 1160 may be arranged proximal to the volume displacement member 1130 and may function in some instances as a distal valve for conveying fluid away from the pump arrangement 1120. One or both of the distal and proximal vascular seals may be configured with one or more features to resist inversion of the valve. For example, the distal vascular seal 1170 may include a member with one or more reinforcement members. As another example, the proximal vascular seal 1160 may include one or more tethers 1162, which may be similar to tethers 762 described above with reference to FIGS. 7A and 7B.

FIG. 12B illustrates an example circulatory assist device 1200 similar to circulatory assist device 1100 described with reference to FIG. 11A except as described below. For example, the circulatory assist device 1200 may include a pump arrangement 1220 with a pump region 1220a configured to receive fluid (e.g., blood). A volume displacement member 1230 may be arranged in the pump region 1220a and configured to drive received fluid towards and through an outflow region of the pump arrangement 920. The volume displacement member 1230 may include a balloon that may be cyclically inflated and deflated with an inflation fluid circulated via one or more lumens in a catheter 1210. A distal vascular seal 1270 may be arranged distal to the volume displacement member 1230 and may function in some instances as an inlet valve for conveying fluid to the pump arrangement 1220. A proximal vascular seal 1260 may be arranged proximal to the volume displacement member 1230 and may function in some instances as a distal valve for conveying fluid away from the pump arrangement 1220. One or both of the distal and proximal vascular seals may be configured with one or more features to resist inversion of the valve. For example, the distal vascular seal 1170 may include a membrane with one or more reinforcement members. As another example, the proximal vascular seal 1260 may include one or more tethers 1262, which may be similar to tethers 762 described above with reference to FIGS. 7A and 7B. However, the circulatory assist device 1200 may include or be combined with a flexible hollow tube 1290, which may enable the circulatory assist device 1200 be operated in non-tubelike cardiovascular lumens, such as a left ventricle. For example, the circulatory assist device 1200 and the tube 1290 may be positioned in a transvalvular location such that a native valve engages with the tube 1290 (e.g., a first portion of the circulatory assist device 1200 and tube 1290 may be positioned in the left ventricle, and a second portion of the circulatory assist device 1200 and tube 1290 may be positioned in the ascending aorta).

FIG. 13 illustrates an example circulatory assist device 1300 including a pump arrangement 1320 with an elongated pump body 1321 having a conduit. The conduit may include an array of inlet valves 1340 configured to convey fluid (e.g., blood) into a pump region 1320a. A volume displacement member 1330 may be arranged in the pump region 1320a and may be configured to drive received fluid towards an outflow region 1320b of the pump arrangement 1320. The volume displacement member 1330 may include a balloon that may be cyclically inflated and deflated with an inflation fluid circulated via one or more lumens in a catheter 1310. The pumped fluid may exit the pump body through an array of outlet valves 1350, which may be radially oriented configured to convey fluid in a radially outward direction, thereby diverging outflow of fluid to a wider outflow profile (e.g., wider cross-sectional outflow profile). The circulatory assist device 1300 as shown in FIG. 13 does not include a vascular seal, but in other variations a vascular seal (e.g., as described herein in accordance with the present technology) may be coupled to or integrally formed with the pump body 1321.

FIG. 14 illustrates an example circulatory assist device 1400 including a pump arrangement 1420 with an elongated pump body 1421 having a conduit. The conduit may include a distal inlet valve 1440 configured to convey fluid (e.g., blood) into a pump region 1420a. A volume displacement member 1430 may be arranged in the pump region 1420a, and may be configured to drive received fluid toward and through an outflow region 1420b of the pump arrangement 1420. The volume displacement member 1430 may include a balloon that may be cyclically inflated and deflated with an inflation fluid circulated via one or more lumens in a catheter 1410.

When the circulatory assist device 1400 is in an active state, the outflow region 1420b may have a maximum expanded diameter that is larger than the pump region 1420a. For example, the outflow region 1420b may have a bulbous shape. A membrane 1424 of the pump body may extend at least partially along the outflow region 1420b (e.g., extending at least to where the outflow region 1020b has its maximum diameter), such that the membrane 1424 and the enlarged shape of the outflow region 1420b may help diverge outflow of fluid to a wider outflow profile. Accordingly, the outflow region 1420b may function as an outflow nozzle in some instances. Additionally or alternatively, the outflow region 1420b may function as a vascular seal, in that in the active expanded state, the outflow region 1420b may be configured to form a peripheral seal against a surrounding lumen wall (e.g., of a descending aorta (DA)).

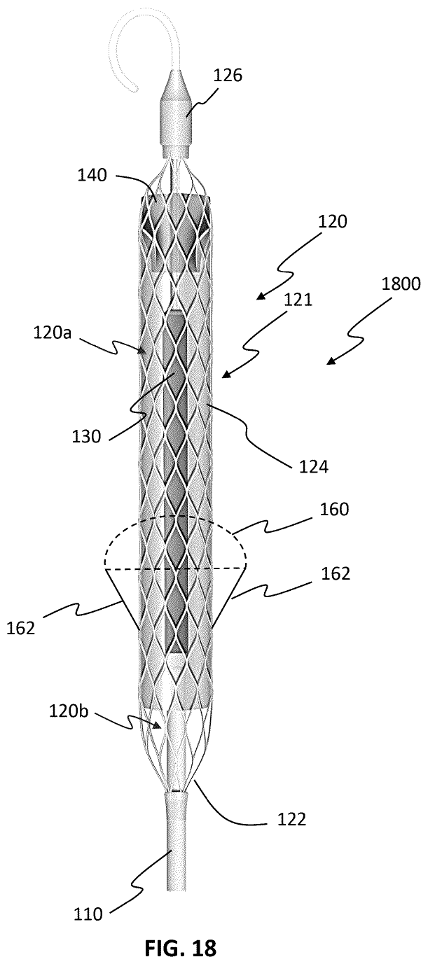

FIG. 18 illustrates an example circulatory assist device 1800 (e.g., an example of circulatory assist device 100). Like the circulatory assist device 100, the circulatory assist device 1800 may include a pump arrangement 120 having a pump body 121 with a pump region 120a configured to receive blood, an outflow region 120b, and a pump 130 in the pump region and configured to drive the received blood toward the outflow region 120b. In some variations, the circulatory assist device 100 may further include a vascular seal 160 configured to form a peripheral seal between the pump arrangement 120 and an inner wall of the aorta (or other cardiovascular lumen such as a pulmonary artery).

As further described herein, the pump body 121 may further include at least one inlet valve 140 configured to receive a fluid through the inlet of the conduit along the flow axis, and a pump 130 arranged in the conduit. The inlet valve 140 may, for example, include a multi-leaflet valve, such as a tri-leaflet valve. Examples of suitable inlet valves for the pump body 121 are described in further detail in U.S. Provisional Patent. Application No. 63/591,900, which is incorporated in its entirety herein by reference. The pump 130 may, for example, include a volume displacement member, impeller, or other suitable pump mechanism. The volume displacement member, if present, may be operable in an expansion phase and a contraction phase. For example, in some variations the volume displacement member may include a balloon, and the balloon may be inflated in the expansion phase, and deflated in the contraction phase. However, the pump may omit an outlet valve that would be configured to convey fluid from the outflow region 120b, away from the pump arrangement 120, and to the cardiovascular lumen.

The circulatory assist device 1800 is characterized by axial flow between the inlet and the outlet of the conduit. In other words, in some variations, fluid pumped by the circulatory assist device 1800 travels from the inlet to the outlet substantially entirely or predominantly axially along (e.g., aligned with) the flow axis of the conduit. In some variations, the fluid flow in the conduit has limited to no radial flow component, and/or limited to no circumferential flow component. The flow axis of the conduit may be substantially coincident with a longitudinal axis of the conduit, for example, though it should be understood that axial flow includes both flow of fluid coincident with the longitudinal axis and flow of fluid generally parallel to the longitudinal axis. The circulatory assist device 1800 with axial flow may have a number of advantages. For example, because forces acting on the fluid within the pump body are generally oriented in the same direction, the fluid travels in a linear path through the circulatory assist device 1800 and experiences less turbulence, thereby resulting in less disturbance in components of the fluid itself (e.g., less hemolysis in blood pumped by the circulatory assist device 1800). Additionally, since flow occurs all in the same general axial direction (e.g., with little to no radial flow component), the kinetic behavior of the pump body (e.g., expansion and contraction of the volume displacement member, such as inflation and deflation of a balloon) can be more streamlined and energy efficient.

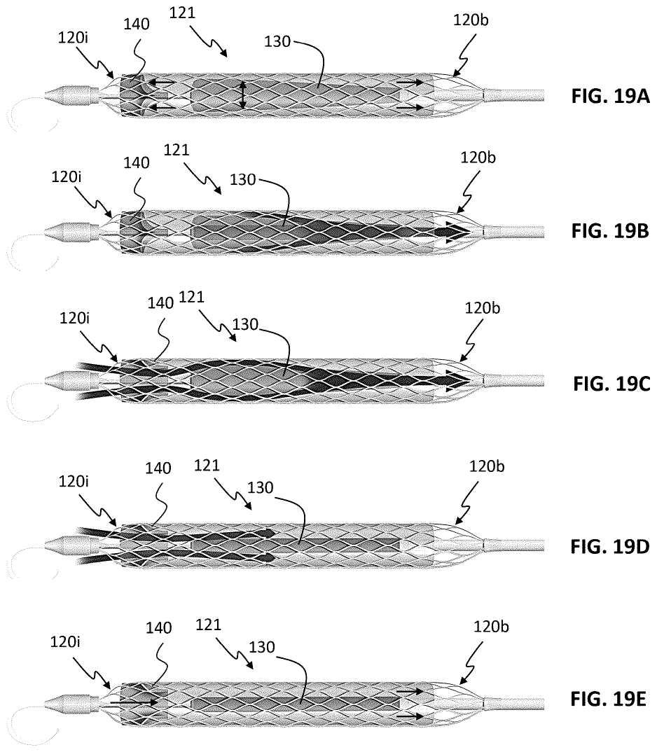

FIGS. 19A-19E illustrate various phases of operation in which fluid may be allowed to exit the conduit of the pump body via maintained momentum during the expansion phase and at least a part of the contraction phase of a pump 130. Although the pump 130 is primarily shown and described below as a balloon, it should be understood that the same principles of operation apply with respect to other variations of circulatory assist devices that include different kinds of pumps 130 (e.g., impeller).

FIG. 19A illustrates a pump body 121 that has received fluid (e.g., blood) through the inlet valve 140, and has a volume displacement member-type pump 130 (e.g., balloon) being inflated to expand within the pump body 121. As the pump 130 inflates, it displaces surrounding fluid, thereby pushing fluid both distally toward the inflow region 120i and proximally toward the outflow region 120b. Fluidic pressure causes the inlet valve 140 to close, while also urging fluid to exit the pump body 121. As shown in FIG. 19B, when the inlet valve 140 is fully closed, all of the fluid volume in the pump body 121 exits through the outlet of the outflow region 120b.

Inflation of the pump 130 also helps generate momentum of the fluid column traveling toward the outflow region 120b in the pump body 121. FIG. 19C illustrates when the pump 130 is at an end portion of the expansion phase, and the pump 130 is inflated to a maximum volume. At this stage of operation, the fluid mass in the pump body 121 has momentum toward the outflow region 120b to exit the pump body 121 through the outlet, and such movement of the fluid mass results in a negative pressure within the pump body 121. Under such momentum and negative pressure within the pump body 121, the inlet valve 140 opens and additional fluid is pulled into the pump body 121 through the open inlet valve 140 in the axial flow direction, as shown in FIG. 19C.

As shown in FIG. 19D, when the pump 130 enters its contraction phase and begins to deflate, the fluid momentum continues, and additional fluid is pulled into the pump body 121 in the axial flow direction through the open inlet valve 140. In some instances, the amount of fluid momentum may decrease at this stage if additional fluid is also pulled into the pump body 121 through the outflow region 120b. However, in these instances, the momentum (and volume) of fluid pulled through the inflow region 120i is greater than that of fluid pulled distally through the outflow region 120b. Accordingly, fluid momentum may slow, but still continues in the direction from the inflow region 120i toward the outflow region 120b, thereby drawing in additional fluid into the pump body 121 for further pumping.

FIG. 19E illustrates when the pump 130 is at an end portion of the contraction phase, and the pump 130 is deflated to a minimum volume. At this stage of operation, the fluid mass continues to have momentum in the proximal direction toward the outflow region 120b, and fluid continues to exit the pump body 121 through the outlet of the conduit. Following the contraction phase, the pump 130 returns to its expansion phase, and the above-described cycle of expansion and contraction (with continued momentum and fluid conveyance through the conduit outlet, as shown in FIGS. 19A-19E) may repeat. Further details regarding axial flow in the circulatory assist device 1800 are described in International Patent Application No. PCT/IB2024/057381, which is incorporated herein in its entirety by reference.

### CONCLUSION

Although many of the variations are described above with respect to systems, devices, and methods for circulatory assistance, the technology is applicable to other applications and/or other approaches. Moreover, other variations in addition to those described herein are within the scope of the technology. Additionally, several other variations of the technology can have different configurations, components, or procedures than those described herein. A person of ordinary skill in the art, therefore, will accordingly understand that the technology can have other variations with additional elements, or the technology can have other variations without several of the features shown and described above with reference to FIGS. 1A-17C.

The descriptions of variations of the technology are not intended to be exhaustive or to limit the technology to the precise form disclosed above. Where the context permits, singular or plural terms may also include the plural or singular term, respectively. Although specific variations of, and examples for, the technology are described above for illustrative purposes, various equivalent modifications are possible within the scope of the technology, as those skilled in the relevant art will recognize. For example, while steps are presented in a given order, alternative variations may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those variations, other variations may also exhibit such advantages, and not all variations need necessarily exhibit such advantages to fall within the scope of the technology. Accordingly, the disclosure and associated technology can encompass other variations not expressly shown or described herein.

I/We claim:

1. A blood pump system configured for placement in a vascular lumen, the blood pump system comprising:
a pump arrangement comprising an outflow region and configured to receive blood and drive the received blood toward the outflow region; and
a vascular seal configured to form a peripheral seal between the pump arrangement and an inner wall of the vascular lumen upstream of the outflow region.

2. The blood pump system of claim 1, wherein the vascular seal is operable in a closed state in which the peripheral seal is formed with the inner wall, and an open state in which blood may flow between the pump arrangement and the inner wall.

3. The blood pump system of claim 2, wherein:
the vascular seal is in the closed state when pressure on a downstream side of the vascular seal is greater than pressure on an upstream side of the vascular seal, and
the vascular seal is in the open state when pressure on the upstream side of the vascular seal is greater than pressure on the downstream side of the vascular seal.

4. The blood pump system of claim 2 or 3, further comprising an actuator configured to actively transition the vascular seal between the closed state and the open state.

5. The blood pump system of any one of claims 2-4, wherein the vascular seal is radially expanded in the closed state and the vascular seal is radially contracted in the open state.

6. The blood pump system of any one of claims 1-5, wherein the pump arrangement comprises a pump body with a conduit.

7. The blood pump system of claim 6, wherein the vascular seal is arranged around a periphery of the pump body.

8. The blood pump system of claim 7, wherein the vascular seal comprises a plurality of segments configured to conform to the inner wall of the vascular lumen.

9. The blood pump system of any of claims 1-8, wherein the vascular seal is located between an inflow region of the pump arrangement and the outflow region.

10. The blood pump system of claim 6 or 9, wherein the conduit comprises a membrane.

11. The blood pump system of claim 10, wherein the conduit further comprises an expandable support, wherein the membrane is adjacent to a surface of the expandable support.

12. The blood pump system of any one of claims 1-11, wherein the pump arrangement further comprises an inlet valve configured to convey blood to the pump region.

13. The blood pump system of any one of claims 1-12, wherein the pump arrangement further comprises an outlet valve configured to convey blood from the outflow region.

14. The blood pump system of any one of claims 1-13, wherein the pump arrangement comprises an impeller pump.

15. The blood pump system of any one of claims 1-13, wherein the pump arrangement comprises a volume displacement member.

16. The blood pump system of claim 15, wherein the volume displacement member comprises a balloon.

17. The blood pump system of claim 16, further comprising:
an inflation member in fluidic communication with the balloon; and
a pump configured to cyclically operate the balloon in an expansion phase and a contraction phase.

18. The blood pump system of any one of claims 1-17, wherein the outflow region comprises a compliant chamber with one or more outlets.

19. The blood pump system of any one of claims 1-18, wherein the vascular seal is configured to extend radially outward from the pump region of the pump arrangement.

20. The blood pump system of any one of claims 1-19, wherein the vascular seal is configured to extend radially outward from the outflow region of the pump arrangement.

21. The blood pump system of any one of claims 1-20, wherein the vascular seal comprises a flexible membrane.

22. The blood pump system of claim 21, wherein the vascular seal comprises one or more reinforcement members coupled to the membrane.

23. The blood pump system of claim 22, wherein the one or more reinforcement members comprises at least one inflatable reinforcement member.

24. The blood pump system of claim 22 or 23, wherein the one or more reinforcement members comprise a shape memory material.

25. The blood pump system of any one of claims 21-24, wherein the membrane comprises one or more flap valves.

26. The blood pump system of any one of claims 1-25, wherein the vascular seal comprises a skirt structure.

27. The blood pump system of any one of claims 1-26, wherein the vascular seal comprises a valve.

28. The blood pump system of any one of claims 1-27, wherein the vascular seal comprises one or more leaflets.

29. The blood pump system of any one of claims 1-28, wherein the outflow region is radially expandable to a bulbous shape to form the vascular seal.

30. The blood pump system of any one of claims 1-29, further comprising one or more tethers configured to control the vascular seal.

31. The blood pump system of any one of claims 1-30, wherein the vascular seal is a first vascular seal, and the blood pump system further comprises a second vascular seal arranged to form a second peripheral seal between the pump arrangement and the inner wall of the vascular lumen.

32. The blood pump system of claim 31, further comprising a conduit containing at least one of the first and second vascular seals.

33. The blood pump system of any one of claims 1-32, wherein the vascular lumen is in a descending aorta.

34. The blood pump system of any one of claims 1-32, wherein the vascular lumen is in a pulmonary artery.

35. A method comprising:
positioning a pump device in a vascular lumen of a patient, the pump device comprising:
a pump arrangement with an outflow region; and
a vascular seal arranged adjacent the pump arrangement;
forming a peripheral seal between the pump arrangement and an inner wall of the vascular lumen with the vascular seal; and
operating the pump arrangement to receive blood and drive the received blood through the outflow region and into the vascular lumen downstream of the peripheral seal.

36. The method of claim 35, wherein positioning the pump device in a vascular lumen comprises positioning the pump device in a descending aorta of the patient.

37. The method of claim 36, further comprising, after operating the pump device in a descending aorta of the patient at a first level of circulatory support, repositioning the pump device at least partially in a left ventricle of the patient and operating the pump device at a second level of circulatory support.

38. The method of claim 37, further comprising, after operating the pump device in the left ventricle of the patient at the second level of the circulatory support, repositioning the pump device in the descending aorta of the patient and operating the pump device at a third level of circulatory support.

39. The method of claim 35, wherein positioning the pump device in a vascular lumen comprises positioning the pump device at least partially in a left ventricle of the patient.

40. The method of claim 39, further comprising, after operating the pump device in the left ventricle at a first level of circulatory support, repositioning the pump device in a descending aorta of the patient and operating the pump device at a second level of circulatory support.

41. The method of claim 35, wherein positioning the pump device in a vascular lumen comprises positioning the pump device in a pulmonary artery of the patient.

42. The method of claim 41, further comprising, after operating the pump device in the pulmonary artery at a first level of circulatory support, repositioning the pump device at least partially in a right ventricle of the patient and operating the pump device at second level of circulatory support.

43. The method of claim 42, further comprising, after operating the pump device in the right ventricle, repositioning the pump device in the pulmonary artery of the patient and operating the pump device at a third level of circulatory support.

44. The method of claim 35, wherein positioning the pump device in a vascular lumen comprises positioning the pump device at least partially in a right ventricle of the patient.

45. The method of claim 44, further comprising, after operating the pump device in the right ventricle at a first level of circulatory support, repositioning the pump device at least partially in a pulmonary artery of the patient at a second level of circulatory support.

46. The method of any one of claims 35-45, wherein the vascular seal is operable in a closed state and an open state.

47. The method of claim 46, wherein the method comprises:
allowing the vascular seal to operate in the closed state when pressure on a downstream side of the vascular seal is greater than pressure in an upstream side of the vascular seal, and
allowing the vascular seal to operate in the open state when pressure on the upstream side of the vascular seal is greater than pressure in the downstream side of the vascular seal.

48. The method of claim 46 or 47, wherein the method comprises actuating the vascular seal to transition the vascular seal between the closed state and the open state

49. The method of any one of claims 35-48, wherein forming a peripheral seal comprises allowing the vascular seal to expand to form the peripheral seal between the pump arrangement and the inner wall of the vascular lumen.

50. The method of claim 49, wherein allowing the vascular seal to expand comprises allowing the vascular seal to self-expand.

51. The method of claim 49, wherein allowing the vascular seal to expand comprises actuating the vascular seal to expand.

52. The method of any one of claims 35-51, wherein the vascular seal comprises a valve.

53. The method of any one of claims 35-52, wherein the vascular seal comprises a skirt structure.

54. The method of any one of claims 35-53, wherein the pump region comprises a pump body with a conduit.

55. The method of any one of claims 35-54, wherein the pump region is defined at least partially by the vascular seal.

56. The method of claim 55, wherein the vascular seal is a first vascular seal, and wherein the pump arrangement is further defined at least partially by a second vascular seal configured to form a second peripheral seal between the pump arrangement and the inner wall of the vascular lumen.

57. The method of any one of claims 35-56, wherein the pump arrangement comprises an impeller pump.

58. The method of any one of claims 35-56, wherein the pump arrangement comprises a volume displacement member.

59. The method of claim 58, wherein the volume displacement member comprises a balloon.

60. The method of any one of claims 35-59, further comprising radially expanding the flow of blood as the blood exits the outflow region.

61. The method of any one of claims 35-60, further comprising actuating one or more actuation members to radially contract the vascular seal, and withdrawing the pump device from the vascular lumen of the patient.

62. The method of any one of claims 35-61, wherein positioning the pump device in the vascular lumen of the patient is performed without imaging guidance.

63. A blood pump system configured for placement in a vascular lumen, the blood pump system comprising:
a pump arrangement comprising:
a conduit with a pump region configured to receive blood and an outflow region; and
a pump in the conduit and configured to drive the received blood through the outflow region,
wherein at least a portion of the pump arrangement is configured to form a peripheral seal with an inner wall of the vascular lumen.

64. The blood pump system of claim 63, wherein the outflow region has an outflow diameter larger than a diameter of the pump region.

65. The blood pump system of claim 63 or 64, wherein the outflow region has a bulbous or flared shape.

66. The blood pump system of claim 64 or 65, wherein the outflow region is configured to form the peripheral seal with the inner wall of the vascular lumen.

67. The blood pump system of any one of claims 64-66, wherein the outflow region is expandable from a contracted shape to an expanded shape having the outflow diameter.

68. The blood pump system of claim 67, wherein the outflow region is self-expandable from the contracted shape to the expanded shape.

69. The blood pump system of claim 67, wherein the outflow region is configured to expand when blood is flowing through the outflow region.

70. The blood pump system of any one of claims 63-69, wherein the conduit comprises at least one inlet region.

71. The blood pump system of claim 70, further comprising at least one one-way valve disposed in the inlet region.

72. The blood pump system of any one of claims 63-71, wherein the conduit comprises one or more radially-directed outlets.

73. The blood pump system of claim 71, wherein the conduit comprises a circumferential array of radially-directed outlets.

74. The blood pump system of any one of claims 63-73, wherein the conduit comprises a membrane.

75. The blood pump system of claim 74, wherein the conduit further comprises an expandable support, wherein the membrane is adjacent to a surface of the expandable support.

76. The blood pump system of any one of claims 63-75, further comprising a vascular seal configured to form the peripheral seal between the pump arrangement and an inner wall of the vascular lumen.

77. The blood pump system of claim 76, wherein the vascular seal comprises a plurality of segments configured to conform to the inner wall of the vascular lumen.

78. The blood pump system of any of claims 63-77, wherein the vascular seal is located between an inflow region of the pump arrangement and the outflow region.

79. The blood pump system of claim 78, wherein the vascular seal is operable in a closed state and an open state.

80. The blood pump system of claim 79, wherein:
the vascular seal is in the closed state when pressure on a downstream side of the vascular seal is greater than pressure on an upstream side of the vascular seal, and
the vascular seal is in the open state when pressure on the upstream side of the vascular seal is greater than pressure on the downstream side of the vascular seal.

81. The blood pump system of claim 79 or 80, further comprising an actuator configured to actively transition the vascular seal between the closed state and the open state.

82. The blood pump system of any one of claims 76-81, wherein the vascular seal comprises a valve.

83. The blood pump system of any one of claims 76-82, wherein the vascular seal comprises a skirt structure.

84. The blood pump system of any one of claims 76-83, wherein the vascular seal comprises a flexible membrane.

85. The blood pump system of claim 84, wherein the vascular seal comprises one or more reinforcement members coupled to the membrane.

86. The blood pump system of claim 85, wherein the one or more reinforcement members comprises at least one inflatable reinforcement member.

87. The blood pump system of claim 85 or 86, wherein the one or more reinforcement members comprise a shape memory material.

88. The blood pump system of any one of claims 63-87, wherein the pump comprises an impeller pump.

89. The blood pump system of any one of claims 63-87, wherein the pump comprises a volume displacement member.

90. The blood pump system of claim 89, wherein the volume displacement member comprises a balloon.

91. The blood pump system of any one of claims 63-90, wherein the vascular lumen is in a descending aorta.

92. The blood pump system of any one of claims 63-90, wherein the vascular lumen is in a pulmonary artery.

93. A method comprising:
positioning a pump device in a vascular lumen of a patient, the pump device comprising a pump arrangement with a conduit having a pump region configured to receive blood and an outflow region, and a pump in the conduit;
operating the pump to drive received blood toward the outflow region; and
engaging an inner wall of the vascular lumen with a sealing portion of the pump device to form a peripheral seal around the pump device.

94. The method of claim 93, wherein the pump device further comprises a vascular seal, wherein the method further comprises forming the peripheral seal between the pump arrangement and an inner wall of the vascular lumen with the vascular seal.

95. The method of claim 93 or 94, wherein the vascular seal comprises the sealing portion of the pump device having an expanded diameter larger than a remaining portion of the pump device.

96. The method of any one of claims 93-95 wherein the sealing portion is adjacent the outflow region of the pump device.

97. The method of claim 95 or 96, further comprising expanding the vascular seal from a contracted diameter to the expanded diameter.

98. The method of claim 97, wherein the vascular seal self-expands from the contracted diameter to the expanded diameter.

99. The method of any one of claims 95-98, wherein the vascular seal expands to the expanded diameter when blood is flowing through the outflow region.

100. The method of any one of claims 94-99, wherein forming a peripheral seal comprises allowing the vascular seal to expand to form the peripheral seal between the pump arrangement and the inner wall of the vascular lumen.

101. The method of claim 100, wherein allowing the vascular seal to expand comprises allowing the vascular seal to self-expand.

102. The method of claim 100, wherein allowing the vascular seal to expand comprises actuating the vascular seal to expand.

103. The method of any one of claims 93-102, wherein the pump comprises an impeller pump.

104. The method of any one of claims 93-102, wherein the pump comprises a volume displacement member.

105. The method of claim 104, wherein the volume displacement member comprises a balloon.

106. The method of any one of claims 93-105, further comprising actuating one or more tethers to radially contract the vascular seal, and withdrawing the pump device from the vascular lumen of the patient.

107. The method of any one of claims 93-106, wherein positioning the pump device in the vascular lumen of the patient is performed without imaging guidance.
